SEQUENCE LISTING (72 sequences)

SEQ ID NO: 1 - protein, 14 aa, Artificial sequence
  Synthetic peptide
RRXCYXKXPYRXCR

SEQ ID NO: 2 - protein, 14 aa, Artificial sequence
  Synthetic peptide
RRXCYXKXPYRXCR

SEQ ID NO: 3 - protein, 14 aa, Artificial sequence
  Synthetic peptide
RRXCYRKXPYRXCR

SEQ ID NO: 4 - protein, 14 aa, Artificial sequence
  Synthetic peptide
RRXCYXKXPYRXCR

SEQ ID NO: 5 - protein, 14 aa, Artificial sequence
  Synthetic peptide
RRXCYXKXPYXXCR

SEQ ID NO: 6 - protein, 14 aa, Artificial sequence
  Synthetic peptide
XRXCYXKXPYRXCR

SEQ ID NO: 7 - protein, 14 aa, Artificial sequence
  Synthetic peptide
XRXCYRKXPYRXCR

SEQ ID NO: 8 - protein, 14 aa, Artificial sequence
  Synthetic peptide
RRXCYRKXPYXXCR

SEQ ID NO: 9 - protein, 14 aa, Artificial sequence
  Synthetic peptide
XRXCYRKXPYXXCR

SEQ ID NO: 10 - protein, 14 aa, Artificial sequence
  Synthetic peptide
RRXCYXKXPYRXCR

SEQ ID NO: 11 - protein, 14 aa, Artificial sequence
  Synthetic peptide
RRXCYXKXPYXXCR

SEQ ID NO: 12 - protein, 14 aa, Artificial sequence
  Synthetic peptide
XRXCYXKXPYRXCR

SEQ ID NO: 13 - protein, 14 aa, Artificial sequence
  Synthetic peptide
XRXCYRKXPYRXCR

SEQ ID NO: 14 - protein, 14 aa, Artificial sequence
  Synthetic peptide
RRXCYRKXPYXXCR

SEQ ID NO: 15 - protein, 14 aa, Artificial sequence
  Synthetic peptide
XRXCYRKXPYXXCR

SEQ ID NO: 16 - protein, 14 aa, Artificial sequence
  Synthetic peptide
XRXCYRKXPYRXCR

SEQ ID NO: 17 - protein, 14 aa, Artificial sequence
  Synthetic peptide
REXCYRKXPYRXCR

SEQ ID NO: 18 - protein, 14 aa, Artificial sequence
  Synthetic peptide
RRXCYEKXPYRXCR

SEQ ID NO: 19 - protein, 14 aa, Artificial sequence
  Synthetic peptide
RRXCYREXPYRXCR

SEQ ID NO: 20 - protein, 14 aa, Artificial sequence
  Synthetic peptide
RRXCYRKXPYRXCR

SEQ ID NO: 21 - protein, 14 aa, Artificial sequence
  Synthetic peptide
RRXCYRKXPYEXCR

SEQ ID NO: 22 - protein, 14 aa, Artificial sequence
  Synthetic peptide
RRXCYRKXPYRXCE

SEQ ID NO: 23 - protein, 14 aa, Artificial sequence
  Synthetic peptide
RRXCYXKXPYRXCR

SEQ ID NO: 24 - protein, 14 aa, Artificial sequence
  Synthetic peptide
RRXCYXKXPYRXCR

SEQ ID NO: 25 - protein, 14 aa, Artificial sequence
  Synthetic peptide
RRXCYXKXPYRXCR

SEQ ID NO: 26 - protein, 14 aa, Artificial sequence
  Synthetic peptide
XRXCYXKXPYRXCR

SEQ ID NO: 27 - protein, 14 aa, Artificial sequence
  Synthetic peptide
RRXCYXKXPXRXCR

SEQ ID NO: 28 - protein, 14 aa, Artificial sequence
  Synthetic peptide
RRXCYXKXPYRXCR

SEQ ID NO: 29 - protein, 14 aa, Artificial sequence
  Synthetic peptide
XRXCYXKXPYRXCR

SEQ ID NO: 30 - protein, 14 aa, Artificial sequence
  Synthetic peptide
RRXCYXKXPXRXCR

SEQ ID NO: 31 - protein, 14 aa, Artificial sequence
  Synthetic peptide
RRXCYXKXPYRXCR

SEQ ID NO: 32 - protein, 14 aa, Artificial sequence
  Synthetic peptide
RRXCYXKXPYRXCR

SEQ ID NO: 33 - protein, 14 aa, Artificial sequence
  Synthetic peptide
RRXCYXKXPYRXCR

SEQ ID NO: 34 - protein, 14 aa, Artificial sequence
  Synthetic peptide
RRXCYXKXPYRXCR

SEQ ID NO: 35 - protein, 13 aa, Artificial sequence
  Synthetic peptide
RXCYXKXPYRXCR

SEQ ID NO: 36 - protein, 14 aa, Artificial sequence
  Synthetic peptide
RRXCYXKXPYRXCR

SEQ ID NO: 37 - protein, 14 aa, Artificial sequence
  Synthetic peptide
RRXCYXKXPYRXCR

SEQ ID NO: 38 - protein, 13 aa, Artificial sequence
  Synthetic peptide
RXCYXKXPYRXCR

SEQ ID NO: 39 - protein, 13 aa, Artificial sequence
  Synthetic peptide
RXCYXKXPYRXCR

SEQ ID NO: 40 - protein, 13 aa, Artificial sequence
  Synthetic peptide
RXCYXKXPYRXCR

SEQ ID NO: 41 - protein, 13 aa, Artificial sequence
  Synthetic peptide
RXCYXKXPYRXCR

SEQ ID NO: 42 - protein, 13 aa, Artificial sequence
  Synthetic peptide
RXCYXKXPYRXCR

SEQ ID NO: 43 - protein, 13 aa, Artificial sequence
  Synthetic peptide
RXCYXKXPYRXCR

SEQ ID NO: 44 - protein, 13 aa, Artificial sequence
  Synthetic peptide
RXCYXKXPYRXCR

SEQ ID NO: 45 - protein, 13 aa, Artificial sequence
  Synthetic peptide
RXCYXKXPYRXCR

SEQ ID NO: 46 - protein, 14 aa, Artificial sequence
  Synthetic peptide
RRXCYXKXPYRXCR

SEQ ID NO: 47 - protein, 14 aa, Artificial sequence
  Synthetic peptide
RRXCYXKXPYRXCR

SEQ ID NO: 48 - protein, 14 aa, Artificial sequence
  Synthetic peptide
RRXCYRKXPYRXCR

SEQ ID NO: 49 - protein, 14 aa, Artificial sequence
  Synthetic peptide
RRXCYXRXPYRXCR

SEQ ID NO: 50 - protein, 14 aa, Artificial sequence
  Synthetic peptide
RRXCYXRXPYRXCR

SEQ ID NO: 51 - protein, 14 aa, Artificial sequence
  Synthetic peptide
RRXCYXKXPYRXCR

SEQ ID NO: 52 - protein, 14 aa, Artificial sequence
  Synthetic peptide
RRXCYRKXPYRXCR

SEQ ID NO: 53 - protein, 14 aa, Artificial sequence
  Synthetic peptide
RRXCYXKXPYRXCR

SEQ ID NO: 54 - protein, 14 aa, Artificial sequence
  Synthetic peptide
RRXCYXKXPYRXCR

SEQ ID NO: 55 - protein, 14 aa, Artificial sequence
  Synthetic peptide
RRXCYXKXPYRXCR

SEQ ID NO: 56 - protein, 14 aa, Artificial sequence
  Synthetic peptide
RRXCYXKXPYRXCR

SEQ ID NO: 57 - protein, 14 aa, Artificial sequence
  Synthetic peptide
ARXCYRKXPYRXCR

SEQ ID NO: 58 - protein, 14 aa, Artificial sequence
  Synthetic peptide
RRXCYAKXPYRXCR

SEQ ID NO: 59 - protein, 14 aa, Artificial sequence
  Synthetic peptide
RRXCYRAXPYRXCR

SEQ ID NO: 60 - protein, 14 aa, Artificial sequence
  Synthetic peptide
RRXCYRKXPYRXCR

SEQ ID NO: 61 - protein, 14 aa, Artificial sequence
  Synthetic peptide
RRXCYRKXAYRXCR

SEQ ID NO: 62 - protein, 14 aa, Artificial sequence
  Synthetic peptide
RRXCYRKXPARXCR

SEQ ID NO: 63 - protein, 14 aa, Artificial sequence
  Synthetic peptide
RRXCYRKXPYAXCR

SEQ ID NO: 64 - protein, 14 aa, Artificial sequence
  Synthetic peptide
XRXCYRKXPYRXCR

SEQ ID NO: 65 - protein, 14 aa, Artificial sequence
  Synthetic peptide
RRXCYXKXPYRXCR

SEQ ID NO: 66 - protein, 14 aa, Artificial sequence
  Synthetic peptide
RRXCYXKXPYRXCR

SEQ ID NO: 67 - protein, 14 aa, Artificial sequence
  Synthetic peptide
RRXCYRXXPYRXCR

SEQ ID NO: 68 - protein, 14 aa, Artificial sequence
  Synthetic peptide
RRXCYXKXPYRXCR

SEQ ID NO: 69 - protein, 14 aa, Artificial sequence
  Synthetic peptide
RRXCYRKXPYRXCR

SEQ ID NO: 70 - protein, 14 aa, Artificial sequence
  Synthetic peptide
RRXCYXKXPYRXCR

SEQ ID NO: 71 - protein, 14 aa, Artificial sequence
  Synthetic peptide
RRXCYRKXPYRXCR

SEQ ID NO: 72 - protein, 14 aa, Artificial sequence
  Synthetic peptide
XRXCYRKXPYRXCR

(19) United States
(12) Patent Application Publication
(10) Pub. No.: US 2013/0303460 A1
(43) Pub. Date: Nov. 14, 2013

(54) PEPTIDES AND COMPOSITIONS FOR THE TREATMENT OF NEUROECTODERMAL DERIVED TUMORS AND RETINOBLASTOMA

(71) Applicant: Amnon Peled, Tel Aviv (IL)

(72) Inventor: Amnon Peled, Tel Aviv (IL)

(73) Assignee: Biokine Therapeutics Ltd., Nes Ziona (IL)

(21) Appl. No.: 13/978,740

(22) PCT Filed: Jan. 10, 2012
(86) PCT No.: PCT/IL12/50008

§ 371 Date: Jul. 9, 2013

Related U.S. Application Data
(60) Provisional application No. 61/431,068, filed on Jan. 10, 2011.
(60) Provisional application No. 61/433,983, filed on Jan. 19, 2011.

(51) Int. Cl. A61K 38/10

(52) U.S. Cl. CPC A61K 38/10
USPC 514/18.9

(57) ABSTRACT

The present invention is directed to compositions and methods for the treatment of retinoblastoma and neuroectodermal derived tumors, such as primitive neuroectodermal tumors (PNET) and neuroblastoma. In particular, the present invention is directed to the use of 4F-benzoyl-TN14003 peptide or analogs or derivatives thereof for treating neuroblastoma and retinoblastoma.

### FIELD OF THE INVENTION

The present invention is directed to compositions and methods for the treatment of retinoblastoma and neuroectodermal derived tumors, such as neuroblastoma.

### BACKGROUND OF THE INVENTION

Primitive neuroectodermal tumors (PNET) are rare tumors usually occurring in children and adolescents. PNETs develop from primitive or undifferentiated neuroepithelial cells from the early development of the nervous system. PNET of the posterior fossa, or medulloblastoma, is the most common brain tumor in children. In 80% of cases, patients with PNETs develop acute hydrocephalus accompanied by severe symptoms of headache and vomiting, and they require urgent resection of the mass (de Bont et al. Exp. Neurol. 2007; 66: 505-516).

Neuroblastoma is the most frequent extra-cranial solid tumor in children, originating from neural crest progenitors cells during embryonic development. In the US, approximately 700 children and adolescents younger than 20 years of age are diagnosed with tumors of the sympathetic nervous system each year, of which approximately 650 are neuroblastomas. Sympathetic nervous system tumors accounted for 7.8% of all cancers among children younger than 15 years of age. Over 97% of sympathetic nervous system tumors are neuroblastomas, embryonal malignancies of the sympathetic nervous system that occur almost exclusively in infants and very young children. Regardless of age, neuroblastomas most commonly occurred in the adrenal gland. The average age at diagnosis is only 23 month, 50% of children are diagnosed at the age of 4-6 years with metastatic disease (stage 4). Prognosis for stage 4 patients is poor, with 75-80% of patients dying 5 years from diagnosis in spite of aggressive treatments.

The clinical presentation of neuroblastoma is highly variable. This has resulted in a dichotomization in therapeutic strategies. For low risk neuroblastoma the trend is to reduce therapeutic intensity. In contrast, the approach to high risk neuroblastoma features intensified chemoradiotherapy combined with surgical tumor removal in order to reach remission, and eradication of minimal residual disease using biological agents such as retinoic acid, immunotherapy and anti-angiogenic therapy. Despite recent advances, 50% to 60% of patients with high-risk neuroblastoma relapse, and to date there are no salvage treatment regimens known to be curative. In light of the unsuccessful therapeutic results in children with advanced stage neuroblastoma, novel therapies are urgently needed.

Retinoblastoma is the most common malignant intraocular tumor in children. In developed countries, the survival rate of children with retinoblastoma is over 95% while in developing countries, due to delayed detection, only 50% of the children survive this tumor. Patients with extraocular retinoblastoma have a poor prognosis for survival, although studies suggest that high dose chemotherapy with stem cell rescue and EBR may be beneficial. Although several treatments are available for retinoblastoma, including chemotherapy, external beam radiotherapy (EBR), and plaque radiotherapy, each of them has major drawbacks in pediatric patients, such as bone marrow suppression, second cancer occurrence, cataracts, retinopathy, and recurrence of the primary tumor.

CXCR4

The chemokine receptor CXCR4 is a G-protein coupled receptor that is expressed in a wide assortment of normal tissues, and plays a fundamental role in fetal development, mobilization of hematopoietic stem cells and trafficking of naive lymphocytes (Rossi and Zlotnik, 2000). The chemokine CXCL12 (also known as stromal-derived factor-1, or SDF-1) is CXCR4's only natural ligand. CXCL12 is expressed constitutively in a variety of tissues, including lung, liver, bone marrow and lymph nodes.

Binding of CXCL12 to CXCR4 activates a variety of intracellular signal transduction pathways and effector molecules that regulate cell chemotaxis, adhesion, survival, and proliferation. For example, the phosphatidyl-inositol-3-kinase pathway and the mitogen-activated protein (MAP) kinase pathways are regulated by CXCL12 and CXCR4.

Various uses of chemokine receptor modulators, including CXCR4 agonists and antagonists, have been described in the art (Princen et al., 2005; Tamamura et al., 2005; U.S. Pat. No. 7,169,750). The bicyclam drug termed AMD3100, originally discovered as an anti-HIV compound, specifically interacts with CXCR4 in an antagonistic manner. Blocking CXCR4 receptor with AMD3100 results in the mobilization of hematopoietic progenitor cells.

US Pub. No. 2007/0167459 discloses heterocyclic compounds having CXCR4 regulating activity, in particular CXCR4 antagonists. These compounds are suggested for the prevention and treatment of various diseases, inter alia a cancerous disease including mammary cancer, malignant lymphoma, cancer metastasis, post-radiotherapy/chemotherapy bone marrow suppression or thrombocytopenia.

U.S. Pat. No. 6,946,445 discloses CXCR4 antagonists comprising the sequence KGVSLSYR. The antagonists disclosed by the '445 patent are suggested to be potentially useful for reducing interferon gamma production by T-cells, treatment of an autoimmune disease, treatment of multiple sclerosis, treatment of other neurological diseases, treatment of cancer, and regulation of angiogenesis. U.S. Pat. No. 6,875,738 discloses methods for treating a solid tumor in a mammal and for inhibiting angiogenesis in a mammal using these antagonists.

U.S. Patent Application Publication No. 2005/0002939 provides the use of antagonists of the CXCR4 protein in diagnosis and therapy of proliferative disease, e.g., ovarian cancer.

T-140 is a 14-residue synthetic peptide developed as a specific CXCR4 antagonist that suppress HIV-1 (X4-HIV-1) entry to T cells through specific binding to CXCR4 (Tamamura et al., 1998). Subsequently, peptide analogs of T-140 were developed as specific CXCR4 antagonist peptides with inhibitory activity at nanomolar levels (see Tamamura et al., 2003, WO 2002/020561 and WO 2004/020462).

WO 2002/020561 discloses novel peptide analogs and derivatives of T-140. The '561 publication demonstrates that the claimed peptides are potent CXCR4 inhibitors, manifesting high anti-HIV virus activity and low cytotoxicity.

WO 2004/020462 discloses additional novel peptide analogs and derivatives of T-140, including 4F-benzoyl-TN14003 (SEQ ID NO: 1). The '462 publication further discloses novel preventive and therapeutic compositions and methods of using same utilizing T-140 analogs for the treatment of cancer and chronic rheumatoid arthritis. The specification of '462 demonstrates the ability of these peptides to inhibit cancer cell migration, including breast cancer and leukemia cells, and to inhibit metastasis formation in vivo. Further demonstrated therein is inhibition of delayed-type hypersensitivity reaction in mice and collagen-induced arthritis, an animal model of rheumatoid arthritis.

WO 2004/087068 is directed to a method for treating or preventing a CXCR4 mediated pathology comprising administering a CXCR4 peptide antagonist to a host in an amount sufficient to inhibit CXCR4 signal transduction in a cell expressing a CXCR4 receptor or homologue thereof, wherein the CXCR4 peptide antagonist is not an antibody or fragment thereof. The '068 publication discloses that exemplary CXCR4 peptide antagonists include T140 and derivatives of T140, and that the pathology includes cancer such as breast, brain, pancreatic, ovarian, prostate, kidney, and non-small lung cancer. Other publications directed to the use of CXCR4 antagonists in cancer therapy include, for example, WO 00/09152, US 2002/0156034, and WO 2004/024178.

WO 08/075,369, to the applicant of the present invention, is directed to therapeutic uses of T-140 analog peptides and compositions comprising same. Particularly, the '369 publication provides compositions and methods for providing improved bone marrow transplantation and in the treatment of other conditions wherein bone marrow depletion or suppression is involved.

WO 08/075,370, to the applicant of the present invention, is directed to novel therapeutic uses of T-140 analog peptides, compositions comprising same, and use thereof useful in cancer therapy.

WO 08/075,371, to the applicant of the present invention, is directed to novel therapeutic uses of T-140 analog peptides, compositions comprising same, and use thereof useful for immunomodulation.

A publication to some of the inventors of the present invention (Avniel et al., 2006) discloses that blocking the CXCR4/CXCL12 axis by a T-140 analog resulted in a significant reduction in eosinophil accumulation in the dermis and improved epithelialization, thus significantly improving skin recovery after burns.

WO 10/146,578, to the applicant of the present invention, provides compositions comprising T-140 analog peptides and methods of use thereof, specifically for providing improved platelet levels useful in the treatment and prevention of thrombocytopenia, for controlling bleeding and for inducing or modulating haemostasis.

WO 10/146,584, to the applicant of the present invention, discloses novel polypeptides comprising a chemokine-binding peptide and an Fc fragment. According to the '584 publication the polypeptides are capable of binding to certain chemokines so as to modulate their activity, and are therefore useful in modulating in vivo chemokine-dependent processes such as inflammation, autoimmunity and cancer.

None of the prior art discloses or suggests that 4F-benzoyl-TN14003 peptide or analogs thereof, specifically stimulate apoptotic cell death of neuroectodermal derived tumors and retinoblastoma cells. There exists a long felt need for compositions and methods useful for treating retinoblastoma and pediatric PNET (e.g., neuroblastoma), in pathological conditions in vivo.

### SUMMARY OF THE INVENTION

The present invention provides compositions and methods for the treatment of retinoblastoma and neuroectodermal derived tumors (e.g., neuroblastoma). Specifically, the present invention provides compositions and methods using 4F-benzoyl-TN14003 peptide or analogs thereof for the treatment of retinoblastoma and neuroblastoma.

The instant invention is based, in part, on the surprising discovery that the known peptide 4F-benzoyl-TN14003 (4F-benzoyl-Arg-Arg-NaI-Cys-Tyr-Cit-Lys-DLys-Pro-Tyr-Arg-Cit-Cys-Arg-NH₂, SEQ ID NO:1) directly and specifically induced apoptotic cell death of neuroblastoma and retinoblastoma, both in vitro and in vivo, thus demonstrating increased anti-tumor effects particularly on tumors of retinoblastoma and neuroectodermal origin.

As exemplified herein below, the 4F-benzoyl-TN14003 peptide did not induce epithelial tumor cell death, and in some cases even stimulated the growth of epithelial tumor cells (e.g., breast and prostate carcinoma and glioblastoma). Surprisingly however, the peptide induced cell death of neuroblastoma and retinoblastoma. Moreover, the anti tumor effect was shown to be unexpectedly exclusive to the peptides of the invention when compared to other CXCR4 antagonists (e.g., AMD 3100). Furthermore, the peptides of the invention demonstrated remarkably significant tumor growth inhibition in vivo.

Thus, the present invention provides method and pharmaceutical compositions for treating a subject having a tumor selected from retinoblastoma and a neuroectodermal derived tumor (e.g., neuroblastoma) in a subject in need thereof, comprising administering to the subject a therapeutically effective amount of a peptide having an amino acid sequence as set forth in SEQ ID NO:1 or an analog or derivative thereof.

In various particular embodiments, the analog or derivative of SEQ ID NO:1 of the methods and pharmaceutical compositions of the invention comprises an amino acid sequence as set forth in the following formula (I) or a salt thereof:

1 2 3 4 5 6 7 8 9 10 11 12 13 14
A₁-A₂-A₃-Cys-Tyr-A₄-A₅-A₆-A₇-A₈-A₉-A₁₀-Cys-A₁₁ (I)

wherein:

A₁ is an arginine, lysine, ornithine, citrulline, alanine or glutamic acid residue or a N-α-substituted derivative of these amino acids, or A₁ is absent;

A₂ represents an arginine or glutamic acid residue if A₁ is present, or A₂ represents an arginine or glutamic acid residue or a N-α-substituted derivative of these amino acids if A₁ is absent;

A₃ represents an aromatic amino acid residue;

A₄, A₅ and A₉ each independently represent an arginine, lysine, ornithine, citrulline, alanine or glutamic acid residue;

A₆ represents a proline, glycine, ornithine, lysine, alanine, citrulline, arginine or glutamic acid residue;

A₇ represents a proline, glycine, ornithine, lysine, alanine, citrulline or arginine residue;

A₈ represents a tyrosine, phenylalanine, alanine, naphthylalanine, citrulline or glutamic acid residue;

A₁₀ represents a citrulline, glutamic acid, arginine or lysine residue;

A₁₁ represents an arginine, glutamic acid, lysine or citrulline residue wherein the C-terminal carboxyl may be derivatized;

and the cysteine residue of the 4-position or the 13-position can form a disulfide bond, and the amino acids can be of either L or D form.

According to certain embodiments, the peptides according to formula (I) are peptides having an amino acid sequence as set forth in any one of SEQ ID NOS:1-72, as presented in Table 1 herein below.

In certain other particular embodiments, said analog or derivative is selected from the group consisting of:

(SEQ ID NO: 1)
4F-benzoyl-Arg-Arg-Nal-Cys-Tyr-Cit-Lys-DLys-Pro-Tyr-Arg-Cit-Cys-Arg-NH₂,
(SEQ ID NO: 2)
Ac-Arg-Arg-Nal-Cys-Tyr-Cit-Lys-DLys-Pro-Tyr-Arg-Cit-Cys-Arg-OH,
(SEQ ID NO: 3)
Ac-Arg-Arg-Nal-Cys-Tyr-Arg-Lys-DCit-Pro-Tyr-Arg-Cit-Cys-Arg-OH,
(SEQ ID NO: 4)
Ac-Arg-Arg-Nal-Cys-Tyr-Cit-Lys-DCit-Pro-Tyr-Arg-Cit-Cys-Arg-OH,
(SEQ ID NO: 10)
Ac-Arg-Arg-Nal-Cys-Tyr-Cit-Lys-DCit-Pro-Tyr-Arg-Cit-Cys-Arg-NH₂,
(SEQ ID NO: 46)
TMguanyl-Arg-Arg-Nal-Cys-Tyr-Cit-Lys-DCit-Pro-Tyr-Arg-Cit-Cys-Arg-NH₂;,
(SEQ ID NO: 47)
ACA-Arg-Arg-Nal-Cys-Tyr-Cit-Lys-DCit-Pro-Tyr-Arg-Cit-Cys-Arg-NH₂,
(SEQ ID NO: 51)
Ac-Arg-Arg-Nal-Cys-Tyr-Cit-Lys-DLys-Pro-Tyr-Arg-Cit-Cys-Arg-NH₂,
(SEQ ID NO: 52)
Ac-Arg-Arg-Nal-Cys-Tyr-Arg-Lys-DCit-Pro-Tyr-Arg-Cit-Cys-Arg-NH₂,
(SEQ ID NO: 53)
4F-benzoyl-Arg-Arg-Nal-Cys-Tyr-Cit-Lys-DGlu-Pro-Tyr-Arg-Cit-Cys-Arg-NHMe,
(SEQ ID NO: 54)
4F-benzoyl-Arg-Arg-Nal-Cys-Tyr-Cit-Lys-DGlu-Pro-Tyr-Arg-Cit-Cys-Arg-NHEt,
(SEQ ID NO: 55)
4F-benzoyl-Arg-Arg-Nal-Cys-Tyr-Cit-Lys-DGlu-Pro-Tyr-Arg-Cit-Cys-Arg-NHiPr,
(SEQ ID NO: 56)
4F-benzoyl-Arg-Arg-Nal-Cys-Tyr-Cit-Lys-DGlu-Pro-Tyr-Arg-Cit-Cys-Arg-tyramine,
(SEQ ID NO: 65)
H-Arg-Arg-Nal-Cys-Tyr-Cit-Lys-DLys-Pro-Tyr-Arg-Cit-Cys-Arg-OH,
(SEQ ID NO: 66)
H-Arg-Arg-Nal-Cys-Tyr-Cit-Lys-DLys-Pro-Tyr-Arg-Cit-Cys-Arg-NH₂,
(SEQ ID NO: 68)
H-Arg-Arg-Nal-Cys-Tyr-Cit-Lys-DCit-Pro-Tyr-Arg-Cit-Cys-Arg-NH₂,
(SEQ ID NO: 70)
H-Arg-Arg-Nal-Cys-Tyr-Cit-Lys-DCit-Pro-Tyr-Arg-Cit-Cys-Arg-OH,
and
(SEQ ID NO: 71)
H-Arg-Arg-Nal-Cys-Tyr-Arg-Lys-DCit-Pro-Tyr-Arg-Cit-Cys-Arg-OH.

According to certain particular embodiments, the peptide is derivatized at the N terminus (i.e., A₁ in formula (I)) with a substituted benzoyl group. In a particular embodiment, the substituted benzoyl group is a 4-fluorobenzoyl group. In another particular embodiment, the substituted benzoyl group is a 2-fluorobenzoyl group. Non limiting examples of peptides derivatized at the N terminus with a substituted benzoyl group are SEQ ID NO: 1, SEQ ID NO: 36-37 and SEQ ID NO:53-SEQ ID NO:56. In a particular exemplary embodiment of said method, the peptide consists of SEQ ID NO: 1.

According to another aspect, the present invention provides a method for treating a subject having a tumor selected from retinoblastoma and a neuroectodermal derived tumor in a subject in need thereof, comprising administering to the subject a therapeutically effective amount of a peptide having an amino acid sequence as set forth in SEQ ID NO:1 or an analog or derivative thereof. The peptides of the invention may be administered to the subject alone or in the form of a pharmaceutical composition comprising the peptide and at least one pharmaceutically acceptable carrier or excipient.

According to another aspect, the present invention provides a pharmaceutical composition comprising a therapeutically effective amount of a peptide comprising an amino acid sequence as set forth in SEQ ID NO:1 or an analog or derivative thereof, and a pharmaceutically acceptable carrier, for the treatment of a tumor selected from retinoblastoma and neuroectodermal derived tumors.

In one embodiment of the methods and composition of the invention, the tumor is retinoblastoma.

In another embodiment of the methods and composition of the invention, the tumor is a neuroectodermal derived tumor. In another embodiment, the neuroectodermal derived tumor is primitive neuroectodermal tumor (PNET) (e.g., neural crest tumor). In certain embodiments, the PNET is selected from peripheral (p)PNET, central nervous system (CNS)PNET and autonomic nervous system PNET. In one embodiment, the PNET is CNS-PNET. In another embodiment, the PNET is pPNET. In another embodiment, the PNET is autonomic nervous system PNET. An exemplary embodiment the autonomic nervous system PNET is neuroblastoma.

In a particular embodiment, the compositions and methods of the present invention are useful in treating pediatric cancers (e.g., brain tumors in children). According to an exemplary embodiment, the pediatric cancer is a pediatric PNET (e.g., neuroblastoma). The term “pediatric”, as used herein, means subjects under the age of 18, preferably 16, more preferably 15.

In another embodiment, the peptides of the invention induce tumor cell death (e.g., apoptosis). In another embodiment, the peptides of the invention inhibit tumor growth. In another embodiment, the peptides of the invention induce tumor cell death and/or reduce tumor growth of a metastasized tumor.

Typically, the peptides of the invention and the pharmaceutical compositions comprising same are administration systemically or locally. In one embodiment, the peptide or the pharmaceutical compositions comprising same is administered locally. In one particular embodiment with respect to treating neuroblastoma, the peptide is administered intra-adrenal. In another particular embodiment with respect to treating retinoblastoma, the peptide is administered by means selected from the group consisting of intraocular, intraorbital, periorbital, ophthalmic or intraconal, wherein each possibility represents a separate embodiment of the invention. Preferably, the peptide is administered intraorbital, wherein the tumor is retinoblastoma.

According to some embodiments, the peptides of the invention are particularly effective in inducing neuroblastoma and/or retinoblastoma cell death when administered in a time-release manner, via, e.g., an implant or a depot. Thus, the peptides or composition comprising same can be, and preferably are, administered in a time-release manner. In one embodiment, the time-release manner is a sustained release. In another embodiment, the time-release manner is a controlled release. Suitable time-release compositions or devices are well known to those of skill in the art. Examples include liposomes, microparticles, microcapsules, and nanoparticles. For instance, the compositions can be prepared from biodegradable polymers, such that the duration of administration can be controlled. In another particular embodiment the pharmaceutical preparation is formulated as a depot for providing controlled or sustained release of the peptide of the invention.

The peptides of the invention may be administered to the subject either alone or in concurrent or sequential combination with other therapeutic agents, including but not limited to chemotherapeutic or other anti-cancer drugs.

According to another aspect, the present invention provides a method for inducing tumor cell death in a subject in need thereof comprising administering to the subject a therapeutically effective amount of a peptide comprising an amino acid sequence as set forth in SEQ ID NO:1 or an analog or derivative thereof, wherein the tumor is selected from the group consisting of retinoblastoma and neuroectodermal derived tumors.

In one embodiment, the tumor is retinoblastoma. In another embodiment, the tumor is retinoblastoma.

According to another aspect, the present invention provides a device for treating a tumor in a subject in need thereof comprising a peptide having an amino acid sequence as set forth in SEQ ID NO:1 or an analog or thereof, formulated for controlled release of the peptide, or an analog thereof, wherein the tumor is selected from the group consisting of retinoblastoma and neuroectodermal derived tumors. In one embodiment, the device is an implant.

Other objects, features and advantages of the present invention will become clear from the following description and drawings.

### BRIEF DESCRIPTION OF THE DRAWINGS

FIGS. 1A-B demonstrate the expression of CXCR4 in retinoblastoma and neuroblastoma tumor cells lines. FIG. 1A shows PCR amplification of CXCR4, CXCL12 and β-actin in Y79 and Weri-Rb1 retinoblastoma tumor cells lines, and in H-SY5Y, SK-N-BE and MHH-NB-11 neuroblastoma tumor cells lines. FIG. 1B is flow cytometric analysis demonstaring CXCR4 expression in retinoblastoma and neuroblastoma tumor cells lines.

FIGS. 2A-B demonstrate the effect of the CXCR4 ligand, CXCL12, on the survival of retinoblastoma cells.

FIGS. 3A-C demonstrate the effect of 4F-benzoyl-TN14003 (designated BKT-140) to stimulate the apoptotic cell death of Y-79 retinoblastoma cells. FIG. 3A depicts the effect of different concentration of BKT-140 (4, 8, 20 and 40) to stimulate retinoblastoma cell death. FIGS. 3B and 3C depicts BKT-140 effect (24 hours) on retinoblastoma survival using FACS analysis.

FIGS. 4A-C demonstrate the effect 4F-benzoyl-TN14003 on the survival of SH-SY5Y, SK-N-BE and MHH-NB-11 neuroblastoma cells (FIGS. 4A, 4B and 4C, respectively).

FIGS. 5A-C demonstrate the effect of CXCR4 antagonists, 4F-benzoyl-TN14003 and AMD 3100, on neuroblastoma cell death in SH-SY5Y, SK-N-BE and MHH-NB-11 cells (FIGS. 5A, 5B and 5C, respectively).

FIGS. 6A-B demonstrate the effect of 4F-benzoyl-TN14003 on neuroblastoma cell death (SH-SY5Y, SK-N-BE and MHH-NB-11 cell lines). FIG. 6A demonstrates neuroblastoma cell death using FACS. FIG. 6B demonstrates neuroblastoma cell death by morphological assays.

FIG. 7 demonstrates 4F-benzoyl-TN14003 tumor growth inhibition effect by injection to mice having neuroblastomsa in a residual disease model.

FIGS. 8A-B demonstrate 4F-benzoyl-TN14003 tumor growth inhibition effect by injection to mice having neuroblastomsa in the adrenal in a treatment disease model.

### DETAILED DESCRIPTION OF THE INVENTION

The present invention provides compositions and methods for the treatment of retinoblastoma and neuroectodermal derived tumors. Specifically, the present invention provides a 4F-benzoyl-TN14003 peptide, or analogs or derivatives thereof, for the treatment of neuroblastoma and retinoblastoma.

The present invention provides in some embodiments, compositions and methods using 4F-benzoyl-TN14003 (as set forth in SEQ ID NO: 1, also known as BKT-140) or analogs or derivatives thereof, useful in the treatment of retinoblastoma and pediatric PNET, as detailed herein. Specifically, there is provided a method for treating a subject having a tumor selected from neuroblastoma and retinoblastoma, comprising administering to the subject a therapeutically effective amount of a peptide having an amino acid sequence as set forth in SEQ ID NO:1 or an analog or derivative thereof.

The 4F-benzoyl-TN14003 analogs used in the novel compositions and methods of the invention (also referred to herein as “the peptides of the invention”) are the structurally and functionally related peptides disclosed in patent applications WO 2002/020561 and WO 2004/020462, also known as “T-140 analogs”, as detailed hereinbelow.

Without wishing to be bound by any theory or mechanism of action, the peptides of the invention are useful for inducing retinoblastoma and neuroectodermal derived tumor cell apoptosis and inhibition of tumor growth.

Peptides

In this specification and drawings, the representations of amino acids, etc. by brevity codes are made by the use of the codes prescribed by IUPAC-IUB Commission on Biochemical Nomenclature or by the codes customarily used in the relevant art. Examples of such codes are shown below. If an optical isomer exists with respect to an amino acid, it preferably represents the L form unless otherwise expressly specified.

Gly or G: glycine; Ala or A: alanine; Val or V: valine; Leu or L: leucine; Ile or I: isoleucine; Ser or S: serine; Thr or T: threonine; Cys or C: cysteine; Met or M: methionine; Glu or E: glutamic acid; Asp or D: aspartic acid; Lys or K: lysine; Arg or R: arginine; H is or H: histidine; Phe or F: phenylalanine; Tyr or Y: tyrosine; Trp or W: tryptophan; Pro or P: proline; Asn or N: asparagine; Gln or Q: glutamine; pGlu: pyroglutamic acid; NaI: 3-(2-naphthyl) alanine; Cit: citrulline; DLys: D-lysine; DCit: D-citrulline; DGlu: D-glutamic acid; Me: methyl group; Et: ethyl group; Bu: butyl group; Ph: phenyl group.

The substituents, protective group and reagents often used in this specification are indicated by the following codes.

| | |
|---|---|
| BHA | benzhydrylamine |
| pMBHA | p-methylbenzhydrylamine |
| Tos | p-toluenesulphonyl |
| CHO | formyl |
| HONB | N-hydroxy-5-norbornene-2,3-dicarboximide |
| OcHex | cyclohexyl ester |
| Bzl | benzyl |
| Cl_{2—}Bzl | dichloro-benzyl |
| Bom | benzyloxymethyl |
| Z | benzyloxycarbonyl |
| Br—Z | 2-bromobenzyloxycarbonyl |
| Boc | t-butyloxycarbonyl |
| DCM | dichloromethane |
| HOBt | 1-hydroxybenzotriazole |
| DCC | N,N′-dicyclohexylcarbodiimide |
| TFA | trifluoroacetic acid |
| DIEA | diisopropylethylamine |
| Fmoc | N-9-fluorenylmethoxycarbony |
| DNP | dinitrophenyl |
| Bum | tertiarybutoxymethyl |
| Trt | trityl |
| Ac | acetyl |
| Guanyl | guanyl |
| Succinyl | succinyl |
| glutaryl | glutaryl |
| TMguanyl | tetramethylguanyl |
| 2F-benzoyl | 2-fluorobenzoyl |
| 4F-benzoyl | 4-fluorobenzoyl |
| APA | 5-aminopentanoyl |
| ACA | 6-aminohexanoyl |
| desamino-Arg | 2-desamino-arginyl |

deamino TMG-APA: the following formula (IV): 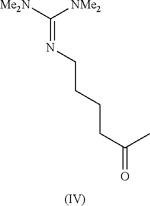
R—CH2: the following formula (V):

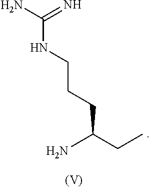

In N-terminal amino acids, [H—] indicates that the terminal amino group is not derivatized, and in C-terminal amino acids, [—OH] indicates that the terminal carboxyl group is not derivatized.

The 4F-benzoyl-TN14003 analogs of the invention belong to a family of structurally closely related peptides, also known as T-140 analogs. T-140 is a known synthetic peptide having the amino acid sequence H-Arg-Arg-NaI-Cys-Tyr-Arg-Lys-DLys-Pro-Tyr-Arg-Cit-Cys-Arg-OH (SEQ ID NO: 69, Tamamura et al., 2003), which was designed based on tachyplesin family polypeptides of the horseshoe crab. The preferable peptides of the invention include analogs and derivatives disclosed in patent applications WO 2002/020561 and WO 2004/020462. These peptides are synthetic peptides of artificial origin.

The term “analog” of SEQ ID NO: 1 as used herein thus relates to a peptide having at least 60% identity to SEQ ID NO: 1, preferably a peptide of Formulae (I) or (II) as defined herein.

In one aspect, the present invention relates to the use of pharmaceutical compositions comprising as an active ingredient a peptide indicated by the following formula (I) or a salt thereof:

1 2 3 4 5 6 7 8 9 10 11 12 13 14
A₁-A₂-A₃-Cys-Tyr-A₄-A₅-A₆-A₇-A₈-A₉-A₁₀-Cys-A₁₁ (I)

wherein:

A₁ in the above-mentioned formula (I) represents an arginine, lysine, ornithine, citrulline, alanine or glutamic acid residue (either L or D form) which may be derivatized at the N-terminus, or A₁ is a hydrogen atom, or it is preferable that A₁ is an arginine, citrulline, alanine or D-glutamic acid residue, or A₁ is a hydrogen atom (i.e. the amino acid at this position may be absent).

Examples of “N-terminal derivatized peptides” or “N-α-substituted derivatives” include, but are not limited to, those protected by formyl group; acyl group, e.g., acetyl group, propionyl group, butyryl group, pentanoyl group, C2-6alkanoyl group e.g. hexanoyl group, benzoyl group, arylcarbonyl group e.g. substituted benzoyl group (e.g.: 2-fluorobenzoyl, 3-fluorobenzoyl group, 4-fluorobenzoyl group, 2-bromobenzoyl group, 3-bromobenzoyl group, 4-bromobenzoyl group, 2-nitrobenzoyl group, 3-nitrobezoyl group, 4-nitrobenzoyl group), succinyl group, glutaryl group; nicotinyl group; isonicotinyl group; alkylsulfonyl group (e.g.: methanesulfonyl group, ethanesulfonyl group, propanesulfonyl group, camphorsulfonyl group); arylsulfonyl group (e.g.: p-toluenesulfonyl group, 4-fluorobenzenesulfonyl group, mesitylenesulfonyl group, 4-aminobenzenesulfonyl group, dansyl group, 4-bromobenzenesulfonyl group) etc. Or, the N-terminal amino acid group may be absent.

A₂ in the above-mentioned formula (I) represents an arginine or glutamic acid residue (either L or D form) if A1 is an arginine, lysine, ornithine, citrulline, alanine or glutamic acid residue (either L or D form) which may be derivatized at the N-terminus, or A₂ represents an arginine or glutamic acid residue (either L or D form) which may be derivatized at the N-terminus if A₁ is absent, or it is preferable that A₂ is an arginine or glutamic acid residue if A₁ is an arginine, citrulline, alanine or glutamic acid residue which may be derivatized at the N-terminus, or A₂ is an arginine or glutamic acid residue which may be derivatized at N-terminus if A₁ is absent. Examples of “peptides derivatized at the N-terminus” include, but are not limited to, the same ones as those mentioned in A1.

A₃ in the above-mentioned formula (I) represents an aromatic amino acid residue (e.g., phenylalanine, tryptophan, 3-(2-naphthyl)alanine, tyrosine, 4-fluorophenylalanine, 3-(1-naphthyl)alanine (either L or D form), or preferably, A₃ represents phenylalanine, tryptophan or 3-(2-naphthyl)alanine.

A₄ in the above-mentioned formula (I) represents an arginine, lysine, ornithine, citrulline, alanine or glutamic acid residue (either L or D form), or it is preferable that A₄ is an arginine, citrulline, alanine or L- or D-glutamic acid residue.

A₅ in the above-mentioned formula (I) represents an arginine, lysine, ornithine, citrulline, alanine or glutamic acid residue (either L or D form), or it is preferable that A₅ is an arginine, citrulline, alanine, lysine or glutamic acid residue.

A₆ in the above-mentioned formula (I) represents a proline, glycine, ornithine, lysine, alanine, citrulline, arginine or glutamic acid residue (either L or D form), or it is preferable that A₆ is a D-lysine, D-alanine, D-citrulline or D-glutamic acid residue.

A₇ in the above-mentioned formula (I) represents a proline, glycine, ornithine, lysine, alanine, citrulline or arginine residue (either L or D form), or it is preferable that A₇ is a proline or alanine residue.

A₈ in the above-mentioned formula (I) represents a tyrosine, phenylalanine, alanine, naphthylalanine, citrulline or glutamic acid residue (either L or D form), or it is preferable that A₈ is a tyrosine, alanine or D-glutamic acid residue.

A₉ in the above-mentioned formula (I) represents an arginine, lysine, ornithine, citrulline, alanine or glutamic acid residue (either L or D form), or it is preferable that A₉ is an arginine, citrulline or glutamic acid residue.

A₁₀ in the above-mentioned formula (I) represents a citrulline, glutamic acid, arginine or lysine residue (either L or D form), or it is preferable that A₁₀ is a citrulline or D-glutamic acid residue.

A₁₁ in the above-mentioned formula (I) represents an arginine, glutamic acid, lysine or citrulline residue (either L or D form) which may be derivatized at C-terminus, or it is preferable that A₁₁ is an arginine or glutamic acid residue which may be derivatized at the C-terminus.

“C-terminal derivatization” or “C-terminal carboxyl derivatization” includes, without limitation, amidation (—CONH₂, —CONHR, —CONRR′) and esterification (—COOR). Herein, R and R′ in amides and esters include, for example, C₁₋₆ alkyl group e.g. methyl, ethyl, n-propyl, isopropyl, or n-butyl, C₃₋₈ cycloalkyl group e.g. cyclopentyl, cyclohexyl, C₆₋₁₂ aryl group e.g. phenyl and a-naphthyl, phenyl-C₁₋₂ alkyl group e.g. benzyl, phenethyl or C₇₋₁₄ aralkyl group e.g. C₁₋₂ alkyl group e.g. a-naphthyl methyl group, and additionally, pivaloyloxymethyl group which is generally used as an oral bioavailable ester.

If a peptide of the present invention has carboxy groups (or carboxylates) at side-chain terminals other than C-terminus, the peptide having amidated or esterificated carboxy groups at side-chain terminals is included in the peptides of the present invention. As the amides and esters in this case, for example, the amides and esters exemplified in A₁₁ are similarly used. Also, the peptides of the present invention include peptides in which substituents (e.g. —OH, —SH, amino group, imidazole group, indole group, guanidino group, etc.) on the intramolecular amino acid side chains are protected by suitable protective group (e.g. C1-6 acyl group, C2-6 alkanoyl such as formyl group, acetyl group, etc.), or complex peptides such as glycopeptides combined with sugar chain in the above-mentioned peptides.

Salts of the peptides of the present invention include physiologically acceptable salts of acids or bases and particularly, physiologically acceptable acid addition salts are preferable. Such salts are exemplified by salts of inorganic acids (e.g. hydrochloric acid, phosphoric acid, hydrobromic acid, sulfuric acid), or salts of organic acids (e.g. acetic acid, formic acid, propionic acid, fumaric acid, maleic acid, succinic acid, tartaric acid, citric acid, malic acid, oxalic acid, benzoic acid, methanesulfonic acid, benzenesulfonic acid).

In one embodiment, the composition comprises a peptide as set forth in formula (I) as defined hereinabove, wherein A₁ is a glutamic acid residue or is absent (not present).

In another embodiment, the composition comprises a peptide as set forth in formula (I) as defined hereinabove, wherein A₄ is a glutamic acid residue.

In another embodiment, the composition comprises a peptide as set forth in formula (I) as defined hereinabove, wherein A₆ is a glutamic acid residue.

In another embodiment, the composition comprises a peptide as set forth in formula (I) as defined hereinabove, wherein A₈ is a glutamic acid residue.

In another embodiment, the composition comprises a peptide as set forth in formula (I) as defined hereinabove, wherein A₉ is a glutamic acid residue.

In another embodiment, the composition comprises a peptide as set forth in formula (I) as defined hereinabove, wherein A₅ is an arginine or glutamic acid residue.

In another embodiment, the composition comprises a peptide as set forth in formula (I) as defined hereinabove, wherein A₁₀ is a glutamic acid, arginine or lysine residue.

In another embodiment, the composition comprises a peptide as set forth in formula (I) as defined hereinabove, wherein A₁₁ is a glutamic acid, lysine or citrulline residue.

In another embodiment, the peptide has an amino acid sequence as set forth in any one of SEQ ID NOS: 1-72 presented in Table 1 herein:

TABLE 1
T-140 and currently preferred T-140 analogs

| | SEQ | |
|---|---|---|
| | ID | |
| Amino acid sequence | NO: | Analog |
| 4F-benzoyl-Arg-Arg-Nal-Cys-Tyr-Cit-Lys-DLys-Pro-Tyr-Arg-Cit-Cys- | 1 | 4F-benzoyl- |
| Arg-NH₂ | | TN14003 |
| Ac-Arg-Arg-Nal-Cys-Tyr-Cit-Lys-DLys-Pro-Tyr-Arg-Cit-Cys-Arg- OH | 2 | AcTC14003 |
| Ac-Arg-Arg-Nal-Cys-Tyr-Arg-Lys-DCit-Pro-Tyr-Arg-Cit-Cys-Arg-OH | 3 | AcTC14005 |
| Ac-Arg-Arg-Nal-Cys-Tyr-Cit-Lys-DCit-Pro-Tyr-Arg-Cit-Cys-Arg-OH | 4 | AcTC14011 |
| Ac-Arg-Arg-Nal-Cys-Tyr-Cit-Lys-DLys-Pro-Tyr-Cit-Cit-Cys-Arg-OH | 5 | AcTC14013 |
| Ac-Cit-Arg-Nal-Cys-Tyr-Cit-Lys-DLys-Pro-Tyr-Arg-Cit-Cys-Arg-OH | 6 | AcTC14015 |
| Ac-Cit-Arg-Nal-Cys-Tyr-Arg-Lys-DCit-Pro-Tyr-Arg-Cit-Cys-Arg-OH | 7 | AcTC14017 |
| Ac-Arg-Arg-Nal-Cys-Tyr-Arg-Lys-DCit-Pro-Tyr-Cit-Cit-Cys-Arg-OH | 8 | AcTC14019 |
| Ac-Cit-Arg-Nal-Cys-Tyr-Arg-Lys-DLys-Pro-Tyr-Cit-Cit-Cys-Arg-OH | 9 | AcTC14021 |
| Ac-Arg-Arg-Nal-Cys-Tyr-Cit-Lys-DCit-Pro-Tyr-Arg-Cit-Cys-Arg-NH₂ | 10 | AcTC14012 |
| Ac-Arg-Arg-Nal-Cys-Tyr-Cit-Lys-DLys-Pro-Tyr-Cit-Cit-Cys-Arg-NH₂ | 11 | AcTC14014 |
| Ac-Cit-Arg-Nal-Cys-Tyr-Cit-Lys-DLys-Pro-Tyr-Arg-Cit-Cys-Arg-NH₂ | 12 | AcTC14016 |
| Ac-Cit-Arg-Nal-Cys-Tyr-Arg-Lys-DCit-Pro-Tyr-Arg-Cit-Cys-Arg-NH₂ | 13 | AcTC14018 |
| Ac-Arg-Arg-Nal-Cys-Tyr-Arg-Lys-DCit-Pro-Tyr-Cit-Cit-Cys-Arg-NH₂ | 14 | AcTC14020 |
| Ac-Cit-Arg-Nal-Cys-Tyr-Arg-Lys-DLys-Pro-Tyr-Cit-Cit-Cys-Arg-NH₂ | 15 | AcTC14022 |
| H-DGlu-Arg-Nal-Cys-Tyr-Arg-Lys-DLys-Pro-Tyr-Arg-Cit-Cys-Arg-OH | 16 | TE14001 |
| H-Arg-Glu-Nal-Cys-Tyr-Arg-Lys-DLys-Pro-Tyr-Arg-Cit-Cys-Arg-OH | 17 | TE14002 |
| H-Arg-Arg-Nal-Cys-Tyr-Glu-Lys-DLys-Pro-Tyr-Arg-Cit-Cys-Arg-OH | 18 | TE14003 |
| H-Arg-Arg-Nal-Cys-Tyr-Arg-Glu-DLys-Pro-Tyr-Arg-Cit-Cys-Arg-OH | 19 | TE14004 |
| H-Arg-Arg-Nal-Cys-Tyr-Arg-Lys-DGlu-Pro-Tyr-Arg-Cit-Cys-Arg-OH | 20 | TE14005 |
| H-Arg-Arg-Nal-Cys-Tyr-Arg-Lys-DLys-Pro-Tyr-Glu-Cit-Cys-Arg-OH | 21 | TE14006 |
| H-Arg-Arg-Nal-Cys-Tyr-Arg-Lys-DLys-Pro-Tyr-Arg-Cit-Cys-Glu-OH | 22 | TE14007 |
| H-Arg-Arg-Nal-Cys-Tyr-Cit-Lys-DGlu-Pro-Tyr-Arg-Cit-Cys-Arg-NH₂ | 23 | TE14011 |
| H-Arg-Arg-Nal-Cys-Tyr-DGlu-Lys-DCit-Pro-Tyr-Arg-Cit-Cys-Arg-NH₂ | 24 | TE14012 |
| H-Arg-Arg-Nal-Cys-Tyr-DGlu-Lys-DGlu-Pro-Tyr-Arg-Cit-Cys-Arg-NH₂ | 25 | TE14013 |
| H-DGlu-Arg-Nal-Cys-Tyr-Cit-Lys-DGlu-Pro-Tyr-Arg-Cit-Cys-Arg-NH₂ | 26 | TE14014 |
| H-Arg-Arg-Nal-Cys-Tyr-Cit-Lys-DGlu-Pro-DGlu-Arg-Cit-Cys-Arg-NH₂ | 27 | TE14015 |
| H-Arg-Arg-Nal-Cys-Tyr-Cit-Lys-DGlu-Pro-Tyr-Arg-DGlu-Cys-Arg-NH₂ | 28 | TE14016 |
| Ac-DGlu-Arg-Nal-Cys-Tyr-Cit-Lys-DGlu-Pro-Tyr-Arg-Cit-Cys-Arg-NH₂ | 29 | AcTE14014 |
| Ac-Arg-Arg-Nal-Cys-Tyr-Cit-Lys-DGlu-Pro-DGlu-Arg-Cit-Cys-Arg- | 30 | AcTE14015 |
| NH₂ | | |
| Ac-Arg-Arg-Nal-Cys-Tyr-Cit-Lys-DGlu-Pro-Tyr-Arg-DGlu-Cys-Arg- | 31 | AcTE14016 |
| NH₂ | | |
| Ac-Arg-Arg-Nal-Cys-Tyr-Cit-Lys-DGlu-Pro-Tyr-Arg-Cit-Cys-Arg-NH₂ | 32 | TF1: |
| | | AcTE14011 |
| guanyl-Arg-Arg-Nal-Cys-Tyr-Cit-Lys-DGlu-Pro-Tyr-Arg-Cit-Cys-Arg- | 33 | TF2: guanyl- |
| NH₂ | | TE14011 |
| TMguanyl-Arg-Arg-Nal-Cys-Tyr-Cit-Lys-DGlu-Pro-Tyr-Arg-Cit-Cys- | 34 | TF3: |
| Arg-NH₂ | | TMguanyl- |
| | | TE14011 |
| TMguanyl-Arg-Nal-Cys-Tyr-Cit-Lys-DGlu-Pro-Tyr-Arg-Cit-Cys-Arg- | 35 | TF4: |
| NH₂ | | TMguanyl- |
| | | TE14011 (2- |
| | | 14) |
| 4F-benzoyl-Arg-Arg-Nal-Cys-Tyr-Cit-Lys-DGlu-Pro-Tyr-Arg-Cit-Cys- | 36 | TF5: 4F- |
| Arg-NH₂ | | benzoyl- |
| | | TE14011 |
| 2F-benzoyl-Arg-Arg-Nal-Cys-Tyr-Cit-Lys-DGlu-Pro-Tyr-Arg-Cit-Cys- | 37 | TF6: 2F- |
| Arg-NH₂ | | benzoyl- |
| | | TE14011 |
| APA-Arg-Nal-Cys-Tyr-Cit-Lys-DGlu-Pro-Tyr-Arg-Cit-Cys-Arg-NH₂ | 38 | TF7: APA- |
| | | TE14011 (2- |
| | | 14) |
| desamino-R-Arg-Nal-Cys-Tyr-Cit-Lys-DGlu-Pro-Tyr-Arg-Cit-Cys-Arg- | 39 | TF8: |
| NH₂ | | desamino-R- |
| | | TE14011 (2- |
| | | 14) |
| Guanyl-Arg-Nal-Cys-Tyr-Cit-Lys-DGlu-Pro-Tyr-Arg-Cit-Cys-Arg-NH₂ | 40 | TF9: guanyl- |
| | | TE14011 (2- |
| | | 14) |
| succinyl-Arg-Nal-Cys-Tyr-Cit-Lys-DGlu-Pro-Tyr-Arg-Cit-Cys-Arg-NH₂ | 41 | TF10: |
| | | succinyl- |
| | | TE14011 (2- |
| | | 14) |
| glutaryl-Arg-Nal-Cys-Tyr-Cit-Lys-DGlu-Pro-Tyr-Arg-Cit-Cys-Arg-NH₂ | 42 | TF11: |
| | | glutaryl- |
| | | TE14011 (2- |
| | | 14) |
| deaminoTMG-APA-Arg-Nal-Cys-Tyr-Cit-Lys-DGlu-Pro-Tyr-Arg-Cit- | 43 | TF12: |
| Cys-Arg-NH₂ | | deaminoTMG- |
| | | APA- |
| | | TE14011 (2- |
| | | 14) |
| R-CH2-Arg-Nal-Cys-Tyr-Cit-Lys-DGlu-Pro-Tyr-Arg-Cit-Cys-Arg-NH₂ | 44 | TF15: H-Arg- |
| | | CH2NH- |
| | | RTE14011 (2- |
| | | 14) |
| H-Arg-Nal-Cys-Tyr-Cit-Lys-DGlu-Pro-Tyr-Arg-Cit-Cys-Arg-NH₂ | 45 | TF17: |
| | | TE14011 (2- |
| | | 14) |
| TMguanyl-Arg-Arg-Nal-Cys-Tyr-Cit-Lys-DCit-Pro-Tyr-Arg-Cit-Cys- | 46 | TF18: |
| Arg-NH₂ | | TMguanyl- |
| | | TC14012 |
| ACA-Arg-Arg-Nal-Cys-Tyr-Cit-Lys-DCit-Pro-Tyr-Arg-Cit-Cys-Arg-NH₂ | 47 | TF19: ACA- |
| | | TC14012 |
| ACA-Arg-Arg-Nal-Cys-Tyr-Arg-Lys-DLys-Pro-Tyr-Arg-Cit-Cys-Arg- | 48 | TF20: ACA- |
| OH | | T140 |
| H-Arg-Arg-Nal-Cys-Tyr-Cit-Arg-DLys-Pro-Tyr-Arg-Cit-Cys-Arg-NH₂ | 49 | TZ14011 |
| Ac-Arg-Arg-Nal-Cys-Tyr-Cit-Arg-DLys-Pro-Tyr-Arg-Cit-Cys-Arg-NH₂ | 50 | AcTZ14011 |
| Ac-Arg-Arg-Nal-Cys-Tyr-Cit-Lys-DLys-Pro-Tyr-Arg-Cit-Cys-Arg-NH₂ | 51 | AcTN14003 |
| Ac-Arg-Arg-Nal-Cys-Tyr-Arg-Lys-DCit-Pro-Tyr-Arg-Cit-Cys-Arg-NH₂ | 52 | AcTN14005 |
| 4F-benzoyl-Arg-Arg-Nal-Cys-Tyr-Cit-Lys-DGlu-Pro-Tyr-Arg-Cit-Cys- | 53 | 4F-benzoyl- |
| Arg-NHMe | | TN14011-Me |
| 4F-benzoyl-Arg-Arg-Nal-Cys-Tyr-Cit-Lys-DGlu-Pro-Tyr-Arg-Cit-Cys- | 54 | 4F-benzoyl- |
| Arg-NHEt | | TN14011-Et |
| 4F-benzoyl-Arg-Arg-Nal-Cys-Tyr-Cit-Lys-DGlu-Pro-Tyr-Arg-Cit-Cys- | 55 | 4F-benzoyl- |
| Arg-NHiPr | | TN14011-iPr |
| 4F-benzoyl-Arg-Arg-Nal-Cys-Tyr-Cit-Lys-DGlu-Pro-Tyr-Arg-Cit-Cys- | 56 | 4F-benzoyl- |
| Arg-tyramine | | TN14011- |
| | | tyramine |
| H-Ala-Arg-Nal-Cys-Tyr-Arg-Lys-DLys-Pro-Tyr-Arg-Cit-Cys-Arg-OH | 57 | TA14001 |
| H-Arg-Arg-Nal-Cys-Tyr-Ala-Lys-DLys-Pro-Tyr-Arg-Cit-Cys-Arg-OH | 58 | TA14005 |
| H-Arg-Arg-Nal-Cys-Tyr-Arg-Ala-DLys-Pro-Tyr-Arg-Cit-Cys-Arg-OH | 59 | TA14006 |
| H-Arg-Arg-Nal-Cys-Tyr-Arg-Lys-DAla-Pro-Tyr-Arg-Cit-Cys-Arg-OH | 60 | TA14007 |
| H-Arg-Arg-Nal-Cys-Tyr-Arg-Lys-DLys-Ala-Tyr-Arg-Cit-Cys-Arg-OH | 61 | TA14008 |
| H-Arg-Arg-Nal-Cys-Tyr-Arg-Lys-DLys-Pro-Ala-Arg-Cit-Cys-Arg-OH | 62 | TA14009 |
| H-Arg-Arg-Nal-Cys-Tyr-Arg-Lys-DLys-Pro-Tyr-Ala-Cit-Cys-Arg-OH | 63 | TA14010 |
| H-Cit-Arg-Nal-Cys-Tyr-Arg-Lys-DLys-Pro-Tyr-Arg-Cit-Cys-Arg-OH | 64 | TC14001 |
| H-Arg-Arg-Nal-Cys-Tyr-Cit-Lys-DLys-Pro-Tyr-Arg-Cit-Cys-Arg-OH | 65 | TC14003 |
| H-Arg-Arg-Nal-Cys-Tyr-Cit-Lys-DLys-Pro-Tyr-Arg-Cit-Cys-Arg-NH₂ | 66 | TN14003 |
| H-Arg-Arg-Nal-Cys-Tyr-Arg-Cit-DLys-Pro-Tyr-Arg-Cit-Cys-Arg-OH | 67 | TC14004 |
| H-Arg-Arg-Nal-Cys-Tyr-Cit-Lys-DCit-Pro-Tyr-Arg-Cit-Cys-Arg-NH₂ | 68 | TC14012 |
| H-Arg-Arg-Nal-Cys-Tyr-Arg-Lys-DLys-Pro-Tyr-Arg-Cit-Cys-Arg-OH | 69 | T-140 |
| H-Arg-Arg-Nal-Cys-Tyr-Cit-Lys-DCit-Pro-Tyr-Arg-Cit-Cys-Arg-OH | 70 | TC14011 |
| H-Arg-Arg-Nal-Cys-Tyr-Arg-Lys-DCit-Pro-Tyr-Arg-Cit-Cys-Arg-OH | 71 | TC14005 |
| H-Cit-Arg-Nal-Cys-Tyr-Arg-Lys-DCit-Pro-Tyr-Arg-Cit-Cys-Arg-NH₂ | 72 | TC14018 |

In each one of SEQ ID NOS: 1-72, two cysteine residues are preferably coupled in a disulfide bond. Currently preferred peptides according to the present invention are peptides having an amino acid sequence as set forth in any one of SEQ ID NOS: 1-72, wherein each possibility represents a separate embodiment of the present invention.

In another particular embodiment, the peptide used in the compositions and methods of the invention consists essentially of an amino acid sequence as set forth in SEQ ID NO: 1. In another preferable embodiment, the peptide used in the compositions and methods of the invention is of an amino acid sequence as set forth in SEQ ID NO:1. In another embodiment, the peptide (analog) is at least 60%, preferably at least 70% and more preferably at least 80% homologous to SEQ ID NO: 1. In another embodiment, the peptide is at least about 90% homologous to SEQ ID NO:1. In another embodiment, the peptide is at least about 95% homologous to SEQ ID NO: 1. Each possibility represents a separate embodiment of the present invention.

It is generally accepted, that the degree of homology between two sequences depends on both the degree of identity in their amino acid sequences and their identity with respect to their length. The peptide homologs of the invention are thus typically about 8-22 amino acids in length, more typically 14-20 amino acid in length or in other embodiments 13-15 amino acids in length, and in particular embodiments about 14 amino acids in length. In various other particular embodiments, the peptide is selected from SEQ ID NOS: 1-72, wherein each possibility represents a separate embodiment of the present invention.

In another particular embodiment, said peptide has an amino acid sequence as set forth in any one of SEQ ID NOS: 1-4, 10, 46, 47, 51-56, 65, 66, 68, 70 and 71. In another particular embodiment, said peptide has an amino acid sequence as set forth in any one of SEQ ID NOS: 4, 10, 46, 47, 68 and 70. In another particular embodiment, said peptide has an amino acid sequence as set forth in any one of SEQ ID NOS: 1, 2, 51, 65 and 66. In another particular embodiment, said peptide has an amino acid sequence as set forth in any one of SEQ ID NOS: 53-56. Each possibility represents a separate embodiment of the invention.

In a preferable particular embodiment, said peptide has an amino acid sequence as set forth in SEQ ID NO: 1. In another particular embodiment, said peptide has an amino acid sequence as set forth in SEQ ID NO:2. In another particular embodiment, said peptide has an amino acid sequence as set forth in SEQ ID NO: 51. In another particular embodiment, said peptide has an amino acid sequence as set forth in SEQ ID NO: 66. Each possibility represents a separate embodiment of the invention.

In another aspect, the invention relates to the use of a pharmaceutical composition comprising a peptide indicated by the following formula (II) or a salt thereof:

1 2 3 4 5 6 7 8 9 10 11 12 13
A₁-Arg-A₂-Cys-Tyr-A₃-A₄-X-A₅-A₆-Cit-Cys-A₇ (II)

wherein:
A₁ represents an arginine, lysine, ornithine, citrulline or alanine residue or an N-α-substituted derivative of these amino acids or a hydrogen atom (namely may be absent);
A₂ represents an aromatic amino acid residue;
A₃, A₄ and A₆ each independently represent an arginine, lysine, ornithine, citrulline or alanine residue;
A₅ represents a tyrosine, phenylalanine, alanine, naphthylalanine or citrulline residue;
A₇ represents a lysine or arginine residue in which a carboxyl group may be amidated or esterified;
X is selected from the group consisting of:
(i) a peptide residue represented by the following formula (III):

1′ 2′ 3′ 4′ 5′ 6′
-A₈-A₉-A₁₀-Gly-A₁₁-A₁₂- (III)

wherein A₈ and Aₗ₂ each independently represents an alanine, valine, leucine, isoleucine, serine, cysteine or methionine residue;
A₉ represents an aromatic amino acid residue, A₁₀ is selected from the same amino acid residues as in A₃, A₁₁ represents a tyrosine, phenylalanine, tryptophan, alanine, valine, leucine, isoleucine, serine, cysteine or methionine residue, provided that when both of the 1′-position and the 6′-position are cysteine residues, they may be bonded in a disulfide bond,
(ii) a peptide selected from the group consisting of a D-ornithyl-proline, prolyl-D-ornithine, D-lysyl-proline, prolyl-D-lysine, D-arginyl-proline, prolyl-D-arginine, D-citrullyl-proline, D-citrullyl-alanine, D-alanyl-citrulline, prolyl-D-citrulline, glycyl-ornithine, ornithyl-glycine, glycyl-lysine, lysyl-glycine, glycyl-arginine, arginyl-glycine, glycyl-citrulline, citrullyl-glycine, D-alanyl-proline, and D-lysyl-alanine,
   and a hydrogen atom of a side chain w-amino group of D-arginine, L-arginine, D-lysine, L-lysine, D-ornithine or L-ornithine which are constitutional amino acids of said peptide residues may be substituted by a ω-aminoacyl group, and the peptide residues of (i) and (ii) represent a peptide residue which binds amino acid residues at the 7-position and the 9-position through a peptide bond;
   and the cysteine residues at the 4-position and the 12-position may be bonded in a disulfide bond;
      provided that, in the above polypeptide or a salt thereof, either of the amino acid residues of A₁, A₃, A₄, A₅, A₆ and A₇ is an alanine or citrulline residue; or
(iii) a peptide residue containing a D-citrulline, D-alanine, citrulline, or alanine residue or a salt thereof.

In the polypeptides of the formula (II) of the present invention, A₁ is preferably an arginine, alanine or citrulline residue; A₂ is preferably a tryptophan or naphthylalanine residue; A₃ is preferably arginine, alanine or citrulline residue; A₄ is preferably a lysine, alanine or citrulline residue; X is preferably a D-lysyl-proline, D-alanyl-proline, D-lysyl-alanine or D-citrullyl-proline residue; A₅ is preferably a tyrosine or alanine residue; A₆ is preferably an arginine, alanine or citrulline residue; A₇ is preferably an arginine residue.

In particular embodiments the peptides of the formula (II) are peptides wherein A₁, A₆ and A₇ are arginine residues, A₂ is a naphthylalanine residue, A₃ is a citrulline residue, A₄ is a lysine residue, X is a D-lysyl-proline residue, and A₅ is a tyrosine residue, a polypeptide of the formula (II) wherein A₁, A₃, A₆ and A₇ are arginine residues, A₂ is a naphthylalanine residue, A₄ is a lysine residue, X is a D-citrullyl-proline residue, and A₅ is a tyrosine residue, a polypeptide of the formula (II) wherein A₁, A₆ and A₇ are arginine residues, A₂ is a naphthylalanine residue, A₃ is a citrulline residue, A₄ is a lysine residue, X is a D-citrullyl-proline residue, A₅ is a tyrosine residue, and a polypeptide of the formula (II) wherein A₁ is a citrulline residue, A₂ is a naphthylalanine residue, A₃, A₆ and A₇ are arginine residues, A₄ is a lysine residue, X is a D-citrullyl-proline residue, A₅ is a tyrosine residue.

The peptides of formula (II) may be exemplified in another embodiment by a peptide of the formula (II) wherein A₁, A₆ and A₇ are arginine residues, A₂ is a naphthylalanine residue, A₃ is a alanine residue, A₄ is a lysine residue, X is a D-lysyl-proline residue, and A₅ is a tyrosine residue, a polypeptide of the formula (II) wherein A₁ is a citrulline residue, A₂ is a naphthylalanine residue, A₃, A₆ and A₇ are arginine residues, A₄ is a lysine residue, X is a D-lysyl-proline residue, and A₅ is a tyrosine residue, a polypeptide of the formula (II) wherein A₁, A₃ and A₇ are arginine residues, A₂ is a naphthylalanine residue, A₄ is a lysine residue, X is a D-lysyl-proline residue, A₅ is a tyrosine residue, and A₆ is a citrulline residue, a polypeptide of the formula (II) wherein A₁ and A₃ are citrulline residues, A₂ is a naphthylalanine residue, A₄ is a lysine residue, X is a D-lysyl-proline residue, A₅ is a tyrosine residue, A₆ and A₇ are arginine residues, and a polypeptide of the formula (II) wherein A₁, A₃ and A₇ are arginine residues, A₂ is a naphthylalanine residue, A₄ is a lysine residue, X is a D-citrullyl-proline residue, A₅ is a tyrosine residue, and A₆ is a citrulline residue.

The amino acid of A₇ as presented in formula II herein is preferably one in which the carboxyl group is amidated for improving stability of the polypeptide in vivo such as in serum, etc.

A peptide of the present invention includes a peptide or its amide, ester or salt containing the amino acid sequence which is substantially the same amino acid sequence as the sequence of any of the above-mentioned peptides. Here, “substantially the same amino acid sequence” means an amino acid sequence that is qualitatively identical in the activity of the peptide or the biological activity of the peptide (e.g. inhibit neuroblastoma and retinoblastoma growth and/or induce their cell death) or the like. Accordingly, quantitative variances are acceptable to some extent (e.g. about 0.01 to 100 times, preferably 0.5 to 20 times, or more preferably 0.5 to 2 times). Therefore, one or more of the amino acids in the amino acid sequences indicated in any of the above-mentioned formula (I), (II) and SEQ ID NOS: 1-72 can have variances, so far as they have any of the above-mentioned properties. That is to say, in the present invention, any peptide (variant peptide) resulting from the variance in the amino acid sequence such as substitution, deletion or insertion (addition) etc. which brings about no significant change (i.e. a qualitatively different change, or a qualitatively identical but quantitatively significantly different change) in the physiological property or chemical property of the original (non-variant) peptide is deemed as substantially the same as the original (non-variant) peptide having no such variance, and, the amino acid sequence of such variant peptide is deemed as substantially the same as the amino acid sequence of the original (non-variant) peptide.

It is a well-known fact that generally, the changes such as substitution, deletion or insertion (addition) of an amino acid in a peptide sequence often do not make a significant change to physiological properties or chemical properties of such peptide. For example, it is generally considered that substitution of a certain amino acid by another amino acid of similar chemical properties results in a peptide having minimized deviation from the properties of the original peptide.

Amino acids are classified, using the similarity of their properties as to one of the criteria, into the following classes, for example: (i) nonpolar (hydrophobic) amino acids (examples: alanine, leucine, isoleucine, valine, proline, phenylalanine, tryptophan, methionine, etc.); (ii) polar (neutral) amino acids (examples: glycine, serine, threonine, cysteine, tyrosine, asparagine, glutamine, etc.); (iii) basic amino acids carrying positive electric charge (examples: arginine, lysine, histidine, etc.); (iv) acidic amino acids carrying negative electric charge (examples: aspartic acid, glutamic acid, etc.), and accordingly, amino acid substitution within each class can be conservative with regard to the property of a peptide (namely, substitution generating “substantially same” amino acid sequences). In other words, “substantially the same amino acid sequences” may include:

(i) amino acid sequences wherein 1 or more, or, in other embodiments, 1 to 3 amino acids were substituted by other amino acids in the amino acid sequences indicated in the above-mentioned formula (I), (II) and SEQ ID NOS:1-72;

(ii) amino acid sequences wherein 1 or more, or, in other embodiments, 1 to 3 amino acids were deleted in the amino acid sequences indicated in the above-mentioned formula (I), (II) and SEQ ID NOS:1-72;

(iii) amino acid sequences wherein 1 or more or, in other embodiments, 1 to 3 amino acids were added (inserted) in the amino acid sequences indicated in the above-mentioned formula (I), (II) and SEQ ID NOS:1-72; or

(iv) peptides including modifications to amino acids (particularly, the side chains thereof) among the peptides having the amino acid sequences indicated in above (i), (ii) or (iii), or esters, amides or salts thereof.

A peptide of the present invention, if and when the substitution, deletion, insertion (addition), modification, etc. of above (i) to (iv) is intentionally or incidentally provided in the amino acid sequence thereof, can be varied to a stable peptide against heat or protease or a high-activity peptide having more enhanced activity. The peptides of the present invention include also these variant peptides or amides thereof, esters thereof or salts thereof.

Furthermore, among the peptides of the present invention are the peptide consisting of the amino acid sequence indicated in any of the above-mentioned formula (I), (II) and SEQ ID NOS:1-72, and the peptide containing the amino acid sequence sharing the homology of about 50 to 99.9% (preferably, 70 to 99.9%, more preferably 90 to 99.9%) with the foregoing amino acid sequence and having the activities of substantially the same nature as the peptide consisting of the amino acid sequence indicated in any of the above-mentioned formula (I), (II) and SEQ ID NOS:1-72, or amides thereof, esters thereof or salts thereof.

Peptide analogs of the invention include in other embodiments peptides which are identical to SEQ ID NO: 1 or other peptides disclosed herein with respect to their amino acid sequence but have different derivatizing groups (e.g. N′ derivatization or C′ derivatization), as long as they are qualitatively identical in their anti-tumor activity as the peptides disclosed herein.

The amides, esters or salts of the peptide having the amino acid sequence indicated in any of the above-mentioned SEQ ID NOS: 1-72 include the same ones as are exemplified for the peptide indicated in the above-mentioned formula (I). Preferably, the peptide having the amino acid sequence indicated in any of the above-mentioned SEQ ID NOS: 1-72 is amidated at the carboxyl group of the C-terminal amino acid residue.

The peptides of the present invention including the peptide containing the amino acid sequence indicated in any of the above-mentioned SEQ ID NOS: 1-72 can be produced by conventionally known methods of synthesizing peptides. For the syntheses of peptides, either solid phase peptide synthesis or liquid phase synthesis may be utilized. Namely, an expected peptide can be produced by condensing a partial peptide able to constitute a peptide or an amino acid with remaining portions, and if the product has a protecting group, by eliminating the protecting group. As the known condensation methods and elimination of protecting groups, the following examples (1) to (5) are included:
(1) M. Bodanszky and M. A. Ondetti, Peptide Synthesis, Interscience Publishers, New York (1966).
(2) Schroeder and Luebke, The Peptide, Academic Press, New York (1965).
(3) N. Izumiya, et. al., Peptide Synthesis, Basics and Practice, Maruzen, Tokyo (1975).
(4) H. Yajima and S. Sakakibara, Seikagaku-Jikken-Koza I, Protein Chemistry IV, Tokyo Kagakudojin, Tokyo, pp 205 (1977).
(5) H. Yajima, Zoku-Iyakuhin-no-Kaihatsu, Vol. 14, Peptide Synthesis, Hirokawa Publishing Co., Tokyo (1991).

As practical methods for syntheses of peptides, the following examples can be given:

Generally, commercially available resins for synthesis of polypeptides can be used. Such resins include, for example, chloromethyl resin, hydroxymethyl resin, benzhydroxylamine resin, aminomethyl resin, 4-hydroxybenzylalcohol resin, 4-methylbenzhydroxylamine resin, PAM resin, 4-hydroxymethylmethylphenylacetoamidomethyl resin, polyacrylamide resin, 4-(2′,4′-dimetoxyphenyl-hydroxymethyl)phenoxy resin, 4-2′,4′-dimetoxyphenyl-Fmoc aminoethylphenoxy resin, etc. Using such resin, an amino acid with suitably protected α-amino group and side chain functional group is condensed on the resin to the sequence of the expected polypeptide in accordance with conventionally known condensation methods. In the last stage of the reaction, the polypeptide is cleared from the resin and simultaneously various protective groups are removed, and then, by carrying out intramolecular disulfide bond-forming reaction in highly diluted solution, the expected polypeptide or amide thereof is obtained. For the above-mentioned condensation of the protected amino acid, various activated reagents usable for the syntheses of polypeptides can be used, but it is particularly better to use carboxylmides. Among such carboxylmides are DCC, N,N′-diisopropylcarbodiimide, N-ethyl-N′-(3-dimethylaminopropyl)cabodiimde, etc. For the activation by these, together with racemization inhibitory additives (for example, HOBt, HOOBt), a protected amino acid is added directly to the resin, or after activating the protected amino acid as symmetric acid anhydride or HOBt ester or HOOBt ester, it can be added to ester resin.

Solvents used for the activation of protected amino acids and the condensation with resins can be chosen from among the solvents known to be usable for polypeptide condensation reactions. For example, acid amides such as N,N-dimethylformamide, N,N-dimethylacetoamide and N-methylpyrrolidone, halogenated hydrocarbons such as methylene chloride and chloroform, alcohols such as trifluoroethanol, sulfoxides such as methyl sulfoxide, ethers such as pyridine, dioxane and tetrahydrofuran, nitriles such as acetonitrile and propionitrile, esters such as methyl acetate and ethyl acetate, or appropriated mixtures of the foregoing are used. A solvent used for activation of a protected amino acid or its condensation with resin can be selected from among the solvents known to be usable for condensing reactions of polypeptides. The reaction temperature is appropriately set within the scope known to be applicable to polypeptide bond forming reactions, usually, at −20° C. to 50° C. Activated amino acid derivatives are usually used at 1.5 to 4 times excess. According to the result of tests adopting ninhydrin reaction, if the condensation is insufficient, the repetition of condensation reactions without eliminating protective groups can lead to sufficient condensation. If sufficient condensation is attained by the repetition of reactions, unreacted amino acids can be acetylated by the use of acetic anhydride or acetylimidazole.

The protective group of the amino group used as ingredients include, for example, Z, Boc, tertialypentyloxycarbony, isobornyloxycarbonyl, 4-methoxybenzyloxycabonyl, Cl—Z, Br—Z, adamantyloxycabonyl, trifluoroacetyl, phtaloyl, formyl, 2-nitrophenylsulphenyl, diphenylphosphinothioyl, Fmoc, etc. Carboxyl group can be protected, for example, by alkyl esterification (e.g. straight-chain, branching or circular alkyl esterification of methyl, ethyl, propyl, butyl, tertialbutyl, cyclopentyl, cyclohexyl, cycloheptyl, cyclooctyl, 2-adamantyl, etc.), aralkyl esterification (e.g. benzylester, 4-nitrobenzylester, 4-methoxybenzylester, 4-chlorbenzylester, benzhydryl esterification), phenacylesterification, benzylcarbonylhydrazidation, tertialybutoxycarbonylhydrazidation, tritylhydrazidation, etc. The hydroxyl group of serine can be protected, for example, by esterification or etherification. The groups suitable for this esterification include, for example, groups derivatized from carboxylic acid such as lower alkanoyl group such as acetyl group, aroyl group such as benzoyl group, benzyloxycarbonyl group, ethoxycarbonyl group. The groups suitable for etherification include, for example, benzyl group, tetrahydropiranyl group, tertiarybutyl group, etc. As the protective groups of phenolic OH group of tyrosine, for example, Bzl, C12-Bzl, 2-nitrobenzyl, Br—Z, tertiarlybutyl, etc. are used. As the protective groups of imidazole of histidine, for example, Tos, 4-methoxy-2,3,6-trimethylbenzenesulfonyl, DNP, benzyloxymethyl, Bum, Boc, Trt, Fmoc etc. are used.

Ingredients with activated carboxyl groups include, for example, corresponding acid anhydride, azide, active ester [ester of alcohol (e.g. pentachlorophenol, 2,4,5-trichlorophenol, 2,4-dinitrophenol, cyanomethylalcohol, p-nitrophenol, HONB, N-hydroxysuccimide, N-hydroxyphtalimide, HOBt)] are used. Ingredients with activated amino group include, for example, corresponding phosphoric amide. As the methods to remove (eliminate) protective groups, for example, catalytic reduction in hydrogen airstream in the presence of a catalyst such as Pd-black or Pd-carbon, acid treatment by anhydrous hydrogen fluoride, methanesulfonic acid, trifluoromethanesulfonic acid, trifluoroacetic acid or a mixture thereof, etc, base treatment by diisopropylethylamine, triethylamine, piperidine, piperadine, etc., and reduction by natrium in liquid ammonia are used. Elimination reaction by the above-mentioned acid treatment is done generally at the temperature of about −20° C. to 40° C., but in the acid treatment, it is effective to add a cation trapping agent such as anisole, phenol, thioanisole, m-cresol, p-cresol, dimethylsulfide, 1,4-butanedithiol, 1,2-ethanedithiol. 2,4-dinitrophenyl group used as the protective group of imidazole of histidine is removed by thiophenol treatment. Formyl group used as the protective group of indole of tryptophan is removed by elimination of protection by the above-mentioned acid treatment in the presence of 1,2-ethanedithiol, 1,4-butanedithiol, etc. and also is removed by alkaline treatment by dilute sodium hydroxide solution, dilute ammonia, etc.

Protection and protective group of functional groups not to be involved in the reaction of ingredients, and elimination of such protective group, and activation of functional groups to be involved in the reaction, etc. can be appropriately selected from among conventionally known groups or conventionally known measures. As alternative methods to obtain amides of polypeptides, there is, for example, a method to manufacture, after amidating and protecting a-carboxyl group of carboxy-terminal amino acid and then extending the peptide chain to the desired chain length on the side of amino group, a polypeptide eliminating the protective group of α-amino group of the N-terminus of such peptide chain and a polypeptide eliminating the protective group of carboxyl group of the C-terminus, and then these two peptides are condensed in the above-mentioned mixed solvent. The details of the condensation reaction are the same as described above. After purifying the protected polypeptide obtained by the condensation, the desired raw polypeptide can be obtained by eliminating all the protective groups by the above-mentioned method. Having purified this raw polypeptide using various known purification methods, if the main fraction is freeze-dried, an amide type of the desired polypeptide can be obtained. To get an ester type of the polypeptide, for example, make an amino acid ester by condensing a-carboxyl group of carboxy-terminal amino acid with the desired alcohols, and then, the ester type of the desired polypeptide can be obtained in the same way as the amide type of the polypeptide.

After the reaction, the peptides of the present invention can be purified and isolated by combining usual purification methods such as solvent extraction, distillation, column chromatography, liquid chromatography, re-crystallization, etc. If a peptide obtained by the above-mentioned methods is a salt-free type, it can be converted to a suitable salt by known methods, or if such peptide is a salt, it can be converted to a salt-free type by known methods.

Pharmaceutical Compositions and Kits

As used herein, a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Hereinafter, the phrases “physiologically acceptable carrier” and “pharmaceutically acceptable carrier”, which may be used interchangeably, refer to a carrier or a diluent that does not cause significant irritation to an organism and does not abrogate the biological activity and properties of the administered compound.

Herein, the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils, and polyethylene glycols.

Techniques for formulation and administration of drugs may be found in the latest edition of “Remington's Pharmaceutical Sciences”, Mack Publishing Co., Easton, Pa., which is herein fully incorporated by reference (Remington: The Science and Practice of Pharmacy, Gennaro, A., Lippincott, Williams & Wilkins, Philadelphia, Pa., 20^{th} ed, 2000).

Pharmaceutical compositions of the present invention may be manufactured by processes well known in the art, e.g., by means of conventional mixing, dissolving, granulating, dragee-making, levigating, emulsifying, encapsulating, entrapping, or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations that can be used pharmaceutically. Proper formulation is dependent upon the route of administration chosen.

The pharmaceutical compositions of the invention are suitable for administration systemically or in a local manner, for example, via injection of the pharmaceutical composition directly into a tissue region of a patient. In a preferred embodiment, the peptide or the pharmaceutical compositions comprising same is administered locally.

For injection, the active ingredients of the pharmaceutical composition may be formulated in aqueous solutions, preferably in physiologically compatible buffers such as Hank's solution, Ringer's solution, or physiological salt buffer.

Pharmaceutical compositions for potential administration include aqueous solutions of the active preparation in water-soluble form. Additionally, suspensions of the active ingredients may be prepared as appropriate oily or water-based injection suspensions. Suitable lipophilic solvents or vehicles include fatty oils such as sesame oil, or synthetic fatty acid esters such as ethyl oleate, triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the active ingredients, to allow for the preparation of highly concentrated solutions.

Alternatively, the active ingredient may be in powder form for constitution with a suitable vehicle, e.g., a sterile, pyrogen-free, water-based solution, before use.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries as desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, and sodium carbomethylcellulose; and/or physiologically acceptable polymers such as polyvinylpyrrolidone (PVP). If desired, disintegrating agents, such as cross-linked polyvinyl pyrrolidone, agar, or alginic acid or a salt thereof, such as sodium alginate, may be added.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions that can be used orally include push-fit capsules made of gelatin, as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate, and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

Alternative embodiments include depots providing sustained release or prolonged duration of activity of the active ingredient in the subject, as are well known in the art.

Pharmaceutical compositions suitable for use in the context of the present invention include compositions wherein the active ingredients are contained in an amount effective to achieve the intended purpose. Determination of a therapeutically effective amount is well within the capability of those skilled in the art, especially in light of the detailed disclosure provided herein. Exemplary doses for human use may be in some embodiments 0.03-10 mg/kg, 0.1-10 mg/kg, 0.1-2 mg/kg, 0.1-1 mg/kg, 0.3-10 mg/kg, 0.3-2 mg/kg, 0.3-1 mg/kg or 0.3-0.9 mg/kg.

The peptides of the current invention derivatives or analogs thereof can be delivered in a controlled release system. Thus, an implant (e.g., an infusion pump) can be used to administer the peptide such as the one that is used, for example, for delivering insulin or chemotherapy to specific organs or tumors. In one embodiment, the peptide of the invention is administered in combination with a biodegradable, biocompatible polymeric implant, which releases the peptide over a controlled period of time at a selected site. Examples of preferred polymeric materials include, but are not limited to, polyanhydrides, polyorthoesters, polyglycolic acid, polylactic acid, polyethylene vinyl acetate, copolymers and blends thereof (See, Medical applications of controlled release, Langer and Wise (eds.), 1974, CRC Pres., Boca Raton, Fla., the contents of which are hereby incorporated by reference in their entirety). In yet another embodiment, a controlled release system can be placed in proximity to a therapeutic target, thus requiring only a fraction of the systemic dose.

In other embodiments, the peptides may be used in combination with anti-cancer treatments, e.g. with one or more chemotherapeutic drugs. In another embodiment, the compositions and methods of the invention enhance the effectiveness of chemotherapy in a subject afflicted with cancer.

In yet another embodiment, the composition consists of a peptide of the invention as a sole active ingredient.

Therapeutic Use

In various embodiments, the peptides of the invention are useful for the treatment of neuroectodermal derived tumor.

In another embodiment, the neuroectodermal derived tumor is primitive neuroectodermal tumor (PNET) (e.g., neural crest tumor). PNET are a group of highly malignant tumors composed of small round cells of neuroectodermal origin that affect soft tissue and bone. PNETs exhibit great diversity in their clinical manifestations and pathologic similarities with other small, round cell tumors. Batsakis et al divided the PNET family of tumors into 3 groups based on the tissue of origin (Batsakis et al. Ann Otol Rhinol Laryngol. 1996; 105(10):838-43):
CNS PNETs—Tumors derived from the central nervous system
Neuroblastoma—Tumors derived from the autonomic nervous system
Peripheral primitive neuroectodermal tumors (pPNETs)—Tumors derived from tissues outside the central and autonomic nervous system

PNET are distinguished into two families based on anatomical location: peripheral (p)PNET and central nervous system (CNS)PNET. The CNS PNET are an heterogeneous group of embryonal tumors including the supratentorial PNET and rare tumors like medulloepithelioma and ependymoblastoma (Raffaghello et al. Semin Cancer Biol 19(2):97-102 2009). In addition, pPNET represent the more differentiated end of a spectrum of neoplasms that comprise: (i) skeletal and extraskeletal Ewing's sarcoma, (ii) peripheral neuroepithelioma, and (iii) neuroblastic tumors (NTs) (Kaatsch P. Cancer Treat Rev (2010) 36(4):277-285). In the adult, melanoma, the deadliest and most frequent form of skin cancer, and small-cell lung carcinoma, also belong to this group of malignancies. The definition of NTs encompasses neuroblastoma stroma-poor (NB), ganglioneuroblastoma (GNB) and ganglioneuroma (GN), which represent three maturational manifestations of a common neoplasm (Schuz et al. J Clin Epidemiol (2001) 54(7):702-709).

In a particular embodiment, the PNET is a pediatric PNET. Pediatric PNET, according to the present invention does not include gliomas, melanomas and small cell carcinoma of the lung. Each possibility is a separate embodiment of the invention.

In one exemplary embodiment, the peptides of the invention are useful for the treatment of neuroblastoma.

Neuroblastoma is a cancer arising in the adrenal gland or less often from the extra-adrenal sympathetic chain, including the retroperitoneum, chest, and neck. Neuroblastoma is the most common cancer among infants. Almost 90% of cases occur in children<5 yr. Neuroblastomas may begin in the abdomen (about 65%), thorax (15 to 20%), neck, pelvis, or other sites. Neuroblastoma occurs very rarely as a primary CNS cancer. Ganglioneuroma is a fully differentiated, benign variant of neuroblastoma. About 40 to 50% of children have localized or regional disease at diagnosis; 50 to 60% have metastases at diagnosis. Neuroblastoma may metastasize to bone marrow, bone, liver, lymph nodes, or, less commonly, skin or brain.

In an additional exemplary embodiment, the peptides of the invention are useful for the treatment of retinoblastoma.

Retinoblastoma is a cancer arising from the immature retina. Symptoms and signs commonly include leukocoria (a white reflex in the pupil), strabismus, and, less often, inflammation and impaired vision. Retinoblastoma occurs in 1/15,000 to 1/30,000 live births and represents about 3% of childhood cancers. It is usually diagnosed in children<2 yr; <5% of cases are diagnosed in those >5 yr.

### EXAMPLES

### Cell Lines

The following human cell lines were used in the study:

Y79: Retinoblastoma cells from primary tumor with familial history of Retinoblastoma;

Weri-Rb1: Retinoblastoma cells from primary tumor;

SH-SY5Y: Neuroblastoma cells from bone marrow derived metastatic tumor;

SK-N-BE: Neuroblastoma cells from bone marrow derived metastatic tumor; and

MHH-NB-11: Neuroblastoma cells from primary tumor.

All cell lines were cultured in RPMI1640 (Gibco BRL life technologies)+10% FCS, 1% L-Glutamine, 1% PS (Penicillin, Streptomycin) (Biological Industries, Kibbutz Beth Haemek, Israel) at 37° C., 5% CO2.

### Example 1

### Expression of CXCR4/CXCL12 Axis in Retinoblastoma and Retinoblastoma Tumors Cell Lines

Total RNA was extracted from various cell lines (Y79, Weri-Rb1, SH-SY5Y, SK-N-BE and MHH-NB-11) using TRIzol reagent (Invitrogen Life Technologies) according to the protocol recommended by manufacture. For cDNA synthesis, 2.5 microgram of total RNA were reverse-transcribed in a final reaction volume of 25 μL containing 1×M-MLV RT buffer, 2.5 μmol/L random hexamers, 0.5 mmol/L each dNTP, 3 mmol/L MgCl₂, 0.4 U/μL RNase inhibitor, and 100 U/μL M-MLV RT. All reverse-transcription (RT) reagents were purchased from Promega, Madison, Wis. The reaction conditions were 1 min at 90° C., 1.5 hour at 42° C., and 15 min at 75° C.

Two microliters of the reverse-transcribed product were subjected to PCR amplification in a final reaction volume of 20 μL containing 1 U of Supertherm Taq polymerase (JMR-Holdings, London, England). Amplification conditions were denaturation at 94° C. for 30 seconds, annealing at 56° C. for 30 seconds, and extension at 72° C. for 30 seconds for 30 consecutive cycles. The PCR amplified products were run on 1% agarose gel containing ethidium bromide. The sizes were estimated by comparison with molecular weight markers.

The following primer pairs were used for PCR:

| | |
|---|---|
| β-actin sense | 5′- CCCTGGACTTCGAGCAAGAG′ -3′, |
| antisense | 5′- TCTCCTTCTGCATCCTGTCG -3′; |
| CXCR4 sense | 5′- AGCTGTTGGCTGAAAAGGTGGTCTATG -3′, |
| antisense | 5′- GCGCTTCTGGTGGCCCTTGGAGTGTG -3′; |
| CXCL12 sense | 5′- ATGAACGCCAAGGTCGTGGTCG -3′, |
| antisense | 5′- TGTTGTTGTTCTTCAGCCG -3′. |

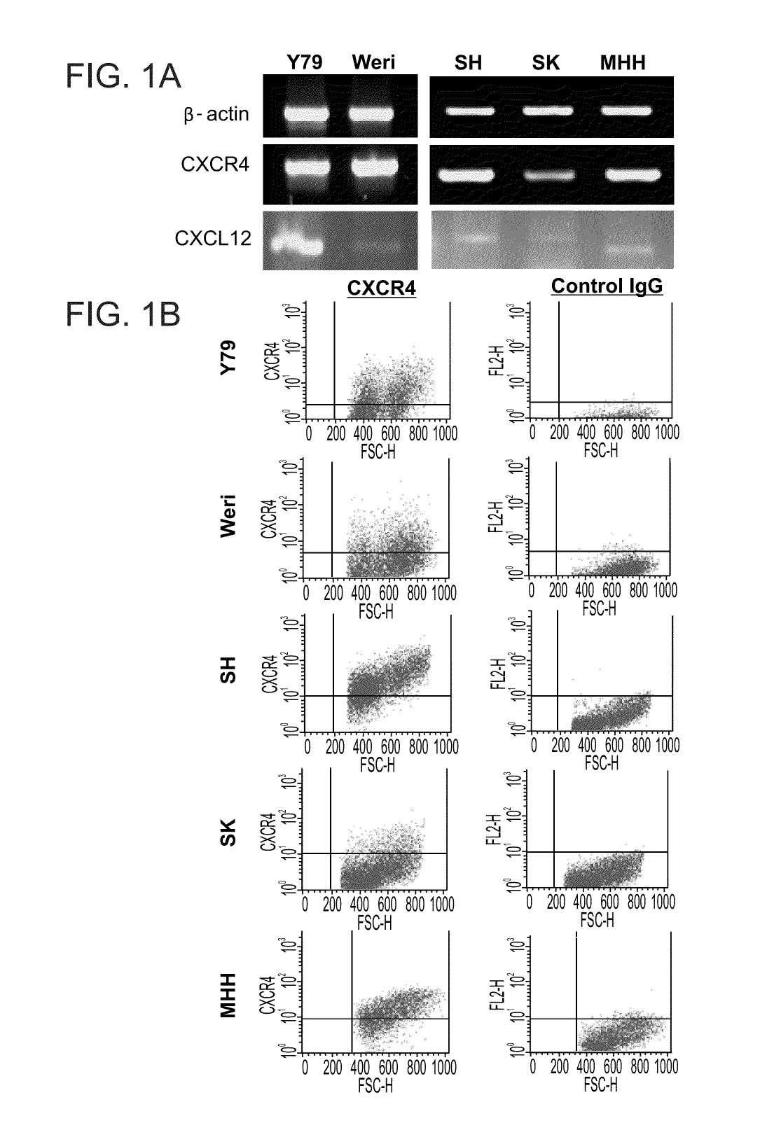

As seen in FIG. 1A, CXCR4, is expressed in Y79 and Weri-Rb1 retinoblastoma tumor cells lines, and in H-SY5Y, SK-N-BE and MHH-NB-11 neuroblastoma tumor cells lines.

Flow Cytometric Analysis

The cells (Y79, Weri-Rb1, SH-SY5Y, SK-N-BE and MHH-NB-11 cell lines) were stained with human specific antibodies and analyzed by FACScalibur (Becton Dickinson), using CellQuest software. For CXCR4 expression analysis, anti-human CXCR4 monoclonal antibody, clone 12G5 (R&D systems) and IgG2A Isotype control monoclonal antibody were used. As seen in FIG. 1B, CXCR4 is expressed in retinoblastoma and neuroblastoma tumor cells lines.

Example 1 indicates that CXCR4 is expressed in various retinoblastoma and retinoblastoma cell lines.

### Example 2

### Effect of CXCL12 on the Survival of Retinoblastoma Cells

Retinoblastoma cells were seeded at 2×10⁴ cells/1 ml per well into a 24-well plate in medium supplemented with 1% FCS with or without various concentrations of CXCL12 (PeproTech EC, London, UK). The cells were incubated for seven days. On day 2, 4 and 7 the attached cells were harvested, stained with PI (Sigma, St. Louis, Mo.), and the number of viable cells was determined using FACS analysis.

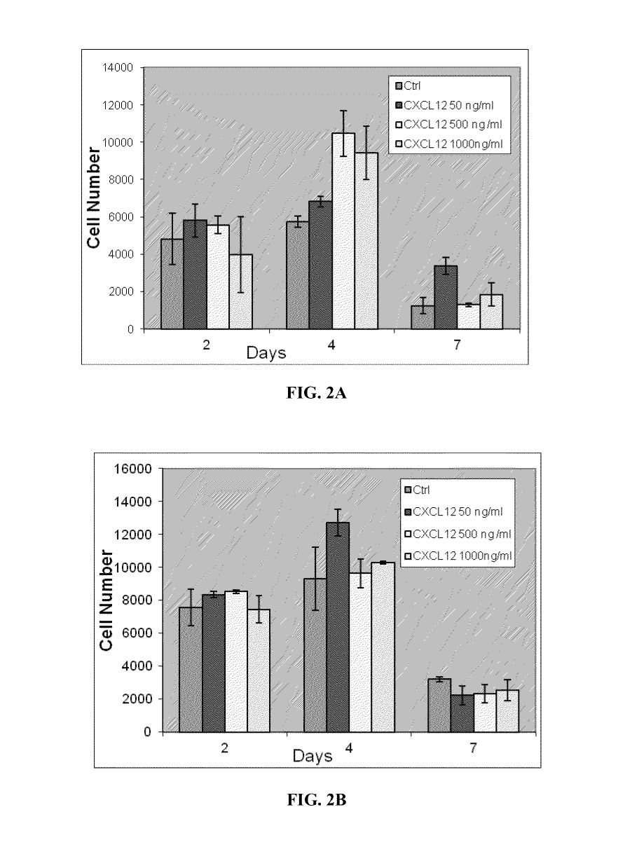

FIG. 2 shows the effect of CXCL12 (50 ng/ml; 500 ng/ml; 1000 ng/ml) on the survival of retinoblastoma cells.

### Example 3

### Effect of the CXCR4 Antagonist 4F-Benzoyl-TN14003 on the Survival of Y79 Retinoblastoma Cells

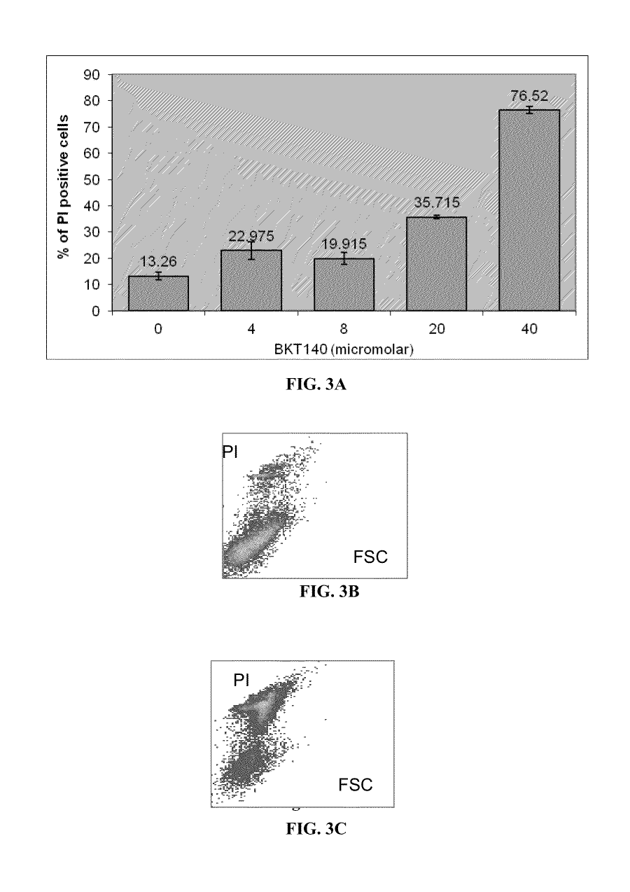

4F-benzoyl-TN14003 (designated BKT140) effect on Y79 Retinoblastoma cells survival. FIG. 3A demonstrates 4F-benzoyl-TN14003 effect at different concentrations (4, 8, 20 and 40 micromolar, 24 hr) on the survival of Y79 cells. FIG. 3B and FIG. 3C demonstrate FACS analysis using PI staining (before treatment and 24 hr following treatment, respectively).

Example 3 shows the effect of CXCR4 antagonist, 4F-benzoyl-TN14003 to stimulate cell death of retinoblastoma cells.

### Example 4

### Effect of the CXCR4 Antagonist 4F-Benzoyl-TN14003 on the Survival of Neuroblastoma Cells

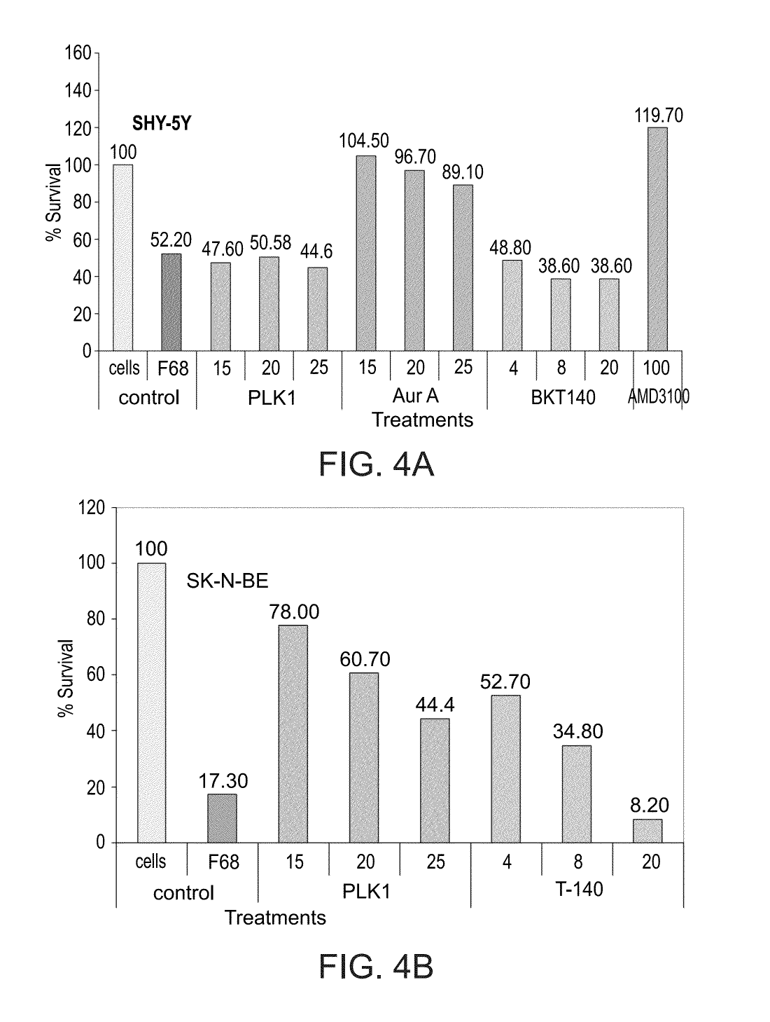

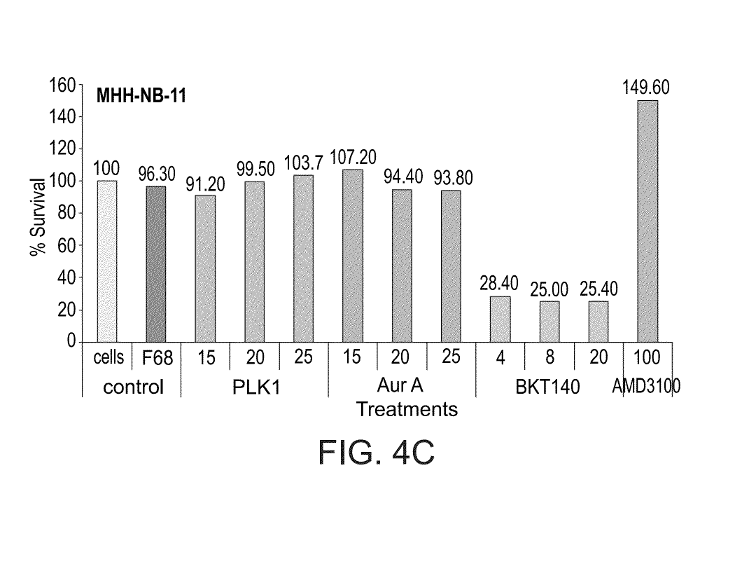

Neuroblastoma cancer cell lines (SHY-5Y, SK-N-BE and NHH-NB-11, FIGS. 4A, 4B, and 4C, respectively) were seeded at 2×10⁴ viable cells/100 μl per well into a 96-well plate in triplicates in a medium supplemented with 10% FCS and incubated with different concentrations of BKT140, PLK1, AurA or AMD3100 for 72 hours. The plate was than tested for cell viability using the CellTiter 96® AQueous Non-Radioactive Cell Proliferation Assay.

Example 4 shows remarkably low cell viability using 4F-benzoyl-TN14003 as compared to other CXCR4 antagonists (e.g. AMD3100).

### Example 5

### The CXCR4 Antagonist 4F-Benzoyl-TN14003 Stimulates Neuroblastoma Cell Death

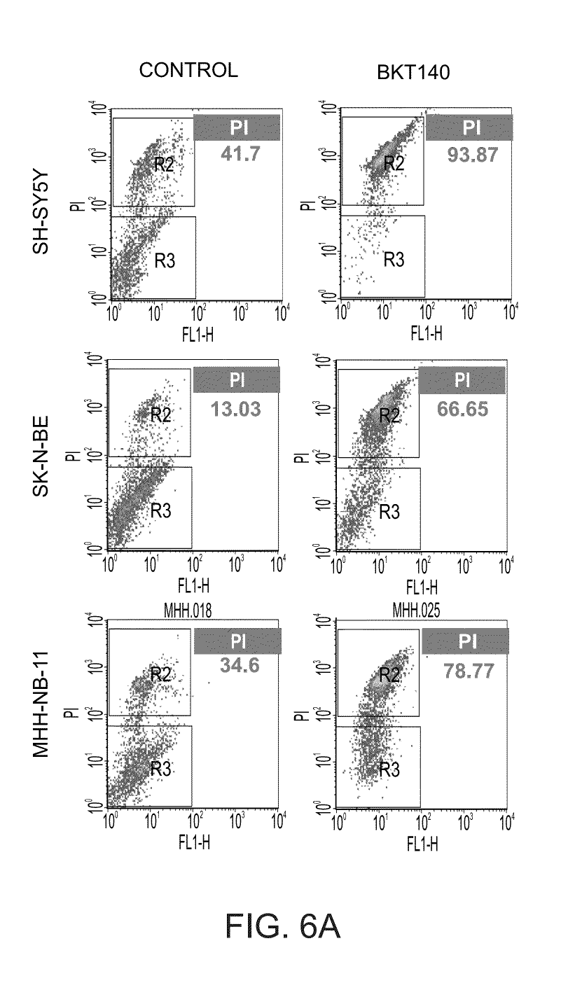

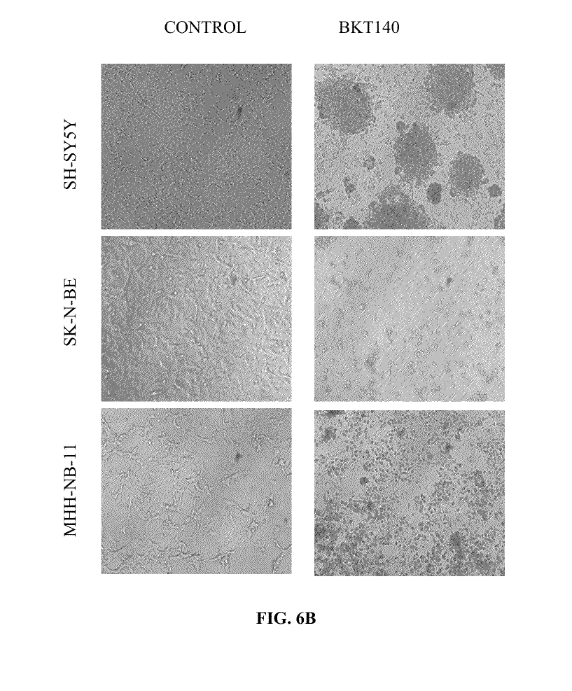

The effect of BKT140 or AMD3100 on the proliferation of neuroblastoma cancer cell lines was tested by seeding the cells at 2×10⁴ cells/1 ml per well into a 24-well plate in medium supplemented with 1% FCS. Twenty hr later the cells stained with PI (Sigma, St. Louis, Mo.), and the number of PI+cells (death cells) was determined using FACS analysis (as described above) (see, FIG. 6A).

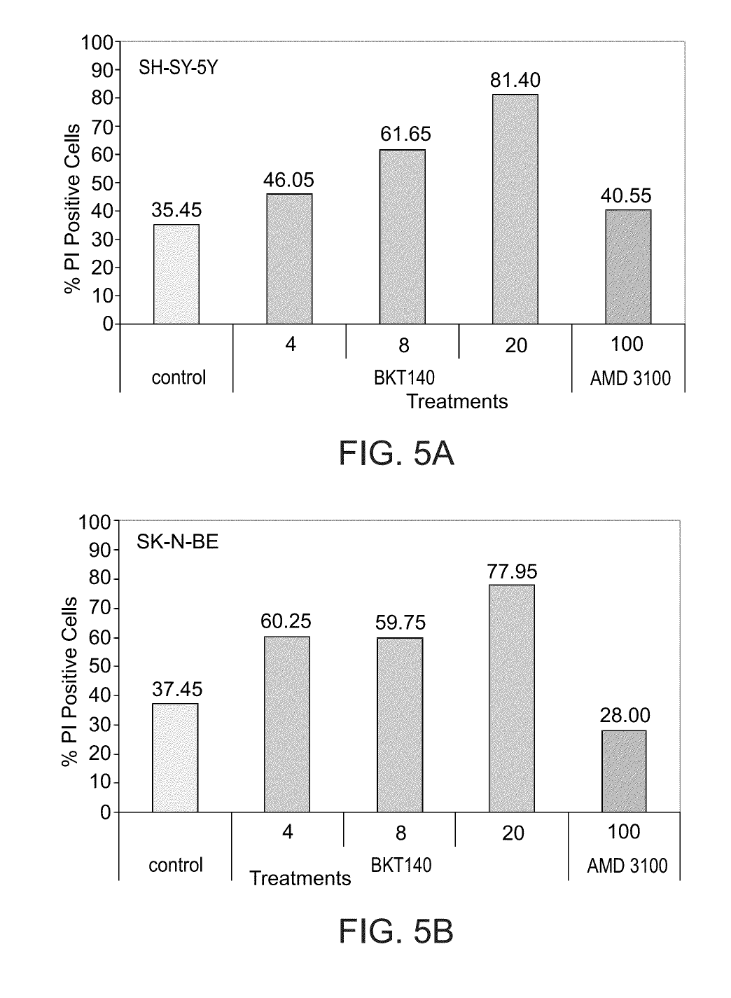

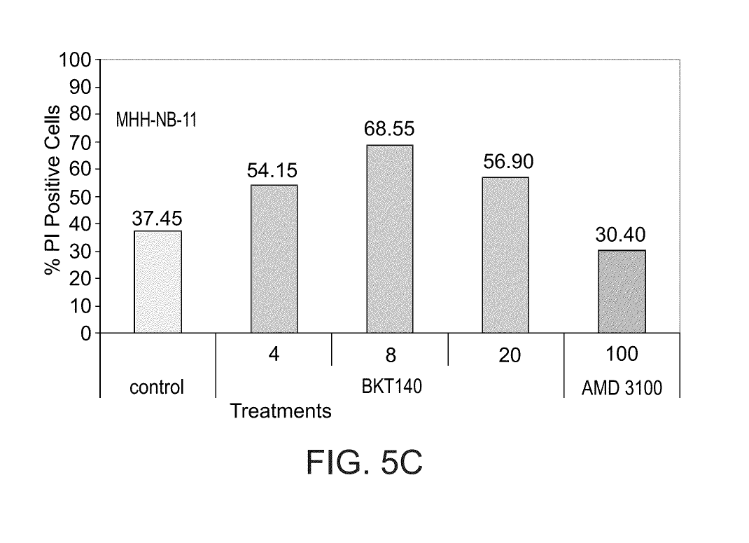

FIG. 5 shows the effect of 4F-benzoyl-TN14003 to stimulate neuroblastoma cell death compared to other CXCR4 antagonists (e.g. AMD3100). Experiments were performed on SHY-5Y, SK-N-BE and NHH-NB-11 cell lines.

FIGS. 6A and B show the effect of 4F-benzoyl-TN14003 to stimulate neuroblastoma cell death (SHY-SY5Y, SK-N-BE and NHH-NB-11).

### Example 6

### The CXCR4 Antagonist 4F-Benzoyl-TN14003 Inhibits Neuroblastoma Tumor Growth (Residual and Established Disease)

NOD/SCID mice were maintained under defined flora conditions at the Hebrew University Pathogen-Free Animal Facility. All experiments were approved by the Animal Care Committee of the Hebrew University. 10⁶ Neuroblastoma tumor cells were injected into the adrenal of the mice (500 μl per mouse). As control, mice were treated with PBS. Tumor growth was monitored weekly. MRI testing was performed on a horizontal 4.7 T Bruker Biospec spectrometer, using a birdcage coil.

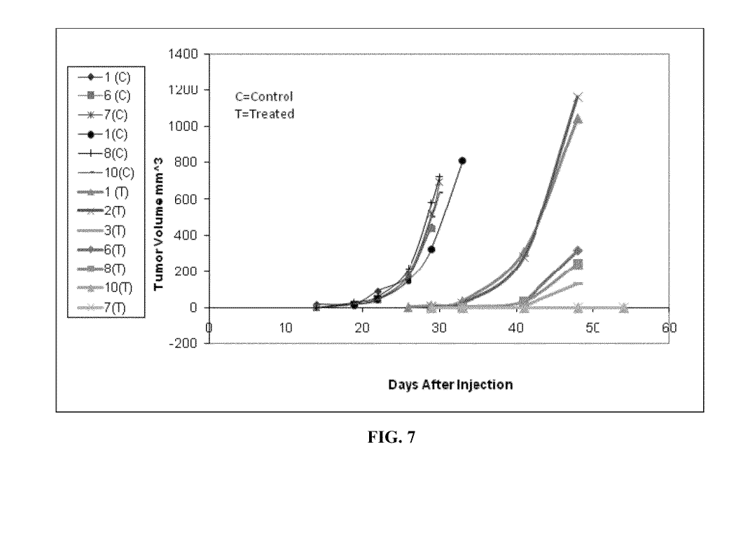

FIG. 7 shows the tumor growth in mice pre treated with 4F-benzoyl-TN14003 (BKT140; 300 μg per mouse) from day 3 until day 30 after inculcation. As seen, 4F-benzoyl-TN14003 delayed and in some cases inhibited tumor growth.

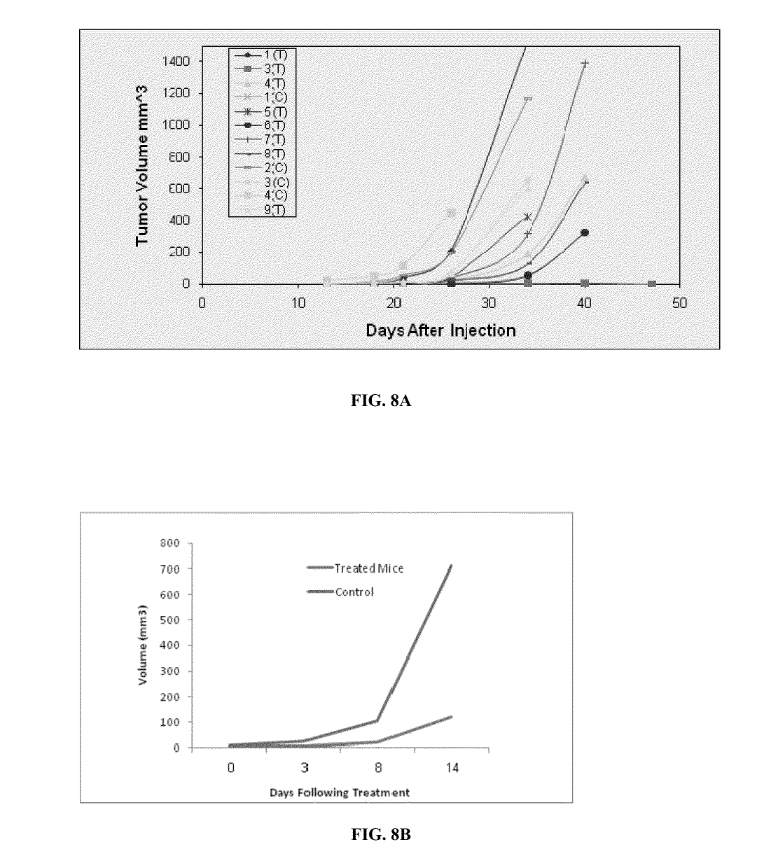

FIG. 8 shows the tumor growth in mice randomized to drug-treated or control PBS-treated groups when the tumor size (width×length) reached ˜0.04 cm² and 4F-benzoyl-TN14003 was administered subcutaneously at a dose of (300 μg per mouse) until day 30. As seen, 4F-benzoyl-TN14003 delayed and in some cases inhibited tumor growth.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A method for treating a subject having a tumor selected from the group consisting of retinoblastoma and neuroectodermal derived tumors, comprising administering to the subject a therapeutically effective amount of a peptide comprising an amino acid sequence as set forth in SEQ ID NO:1 or an analog or derivative thereof.

2. The method of claim 1, wherein the analog or derivative comprises an amino acid sequence as set forth in formula (I) or a salt thereof:
1 2 3 4 5 6 7 8 9 10 11 12 13 14
A₁-A₂-A₃-Cys-Tyr-A₄-A₅-A₆-A₇-A₈-A₉-A₁₀-Cys-A₁₁ (I)
wherein:
A₁ is an arginine, lysine, ornithine, citrulline, alanine or glutamic acid residue or a N-α-substituted derivative of these amino acids, or A₁ is absent;
A₂ represents an arginine or glutamic acid residue if A₁ is present, or A₂ represents an arginine or glutamic acid residue or a N-α-substituted derivative of these amino acids if A₁ is absent;
A₃ represents an aromatic amino acid residue;
A₄, A₅ and A₉ each independently represents an arginine, lysine, ornithine, citrulline, alanine or glutamic acid residue;
A₆ represents a proline, glycine, ornithine, lysine, alanine, citrulline, arginine or glutamic acid residue;
A₇ represents a proline, glycine, ornithine, lysine, alanine, citrulline or arginine residue;
A₈ represents a tyrosine, phenylalanine, alanine, naphthylalanine, citrulline or glutamic acid residue;
A₁₀ represents a citrulline, glutamic acid, arginine or lysine residue;
A₁₁ represents an arginine, glutamic acid, lysine or citrulline residue wherein the C-terminal carboxyl may be derivatized;
and the cysteine residue of the 4-position or the 13-position can form a disulfide bond, and the amino acids can be of either L or D form.

3. The method of claim 1, wherein the peptide is selected from the group consisting of SEQ ID NOS: 1-72.

4. The method of claim 1, wherein the peptide is derivatized at the N terminus with a substituted benzoyl group.

5. The method of claim 4, wherein the peptide is selected from the group consisting of SEQ ID NO: 1, SEQ ID NO: 36-37 and SEQ ID NO: 53-56.

6. The method of claim 5, wherein the peptide consists of SEQ ID NO: 1.

7. The method of claim 1, wherein the tumor is a neuroectodermal derived tumor.

8. The method of claim 7, wherein the neuroectodermal derived tumor is a primitive neuroectodermal tumor (PNET).

9. The method of claim 8, wherein the PNET is neuroblastoma.

10. The method of claim 8, wherein the PNET is pediatric PNET.

11. The method of claim 7, wherein the peptide is administered intra-adrenally.

12. The method of claim 1, wherein the tumor is retinoblastoma.

13. The method of claim 12, wherein the peptide is administered intraorbitally.

14. The method of claim 1, wherein the peptide is administered in a time-release manner.

15. The method of claim 1, wherein the peptide induces said tumor cell death.

16. The method of claim 1, wherein the peptide inhibits said tumor growth.

17. A pharmaceutical composition comprising a therapeutically effective amount of a peptide comprising an amino acid sequence as set forth in SEQ ID NO:1 or an analog or derivative thereof, and a pharmaceutically acceptable carrier, for the treatment of a tumor selected from retinoblastoma and pediatric primitive neuroectodermal tumors (PNET).

18-28. (canceled)

29. A method for inducing tumor cell death in a subject in need thereof comprising administering to the subject a therapeutically effective amount of a peptide comprising an amino acid sequence as set forth in SEQ ID NO:1 or an analog or derivative thereof, wherein the tumor is selected from the group consisting of retinoblastoma and neuroectodermal derived tumors.

30. The method of claim 29, wherein the peptide is selected from the group consisting of SEQ ID NOS: 1-72.

31. The method of claim 30, wherein the peptide is derivatized at the N terminus with a substituted benzoyl group.

32. The method of claim 31, wherein the peptide is selected from the group consisting of SEQ ID NO: 1, SEQ ID NO: 36-37 and SEQ ID NO: 53-56.

33. The method of claim 32, wherein the peptide consists of SEQ ID NO: 1.

34. The method of claim 29, wherein the tumor is a neuroectodermal derived tumor.

35. The method of claim 34, wherein the neuroectodermal derived tumor is neuroblastoma.

36. The method of claim 29, wherein the tumor is retinoblastoma.
